(19) United States
(12) Patent Application Publication
(10) Pub. No.: US 2025/0368804 A1
(43) Pub. Date: Dec. 4, 2025

(54) EPOXY BIOBASED PLASTICIZERS, METHODS OF PREPARATION AND METHODS OF USE THEREOF

(71) Applicant: Valtris Specialty Chemicals, Independence, OH (US)

(72) Inventors: Mark Holt, Independence, OH (US); Amanda Freshwater, Independence, OH (US); Megan Kravec, Independence, OH (US)

(21) Appl. No.: 19/223,494

(22) Filed: May 30, 2025

Related U.S. Application Data
(60) Provisional application No. 63/654,664, filed on May 31, 2024.

(51) Int. Cl. C08K 5/1515; B60C 1/00; B60C 11/00; C07D 303/38

(52) U.S. Cl. CPC C08K 5/1515; B60C 1/0016; B60C 11/0008; C07D 303/38; B60C 2011/0025

(57) ABSTRACT

Disclosed herein are biobased epoxy soyates and carboxylates, compositions containing such soyates and carboxylates, methods of preparation and methods of use thereof. Disclosed is an epoxy soyate of Formula (I): (R—O)ₙ—R₁ (I) wherein n is an integer from 1 to 4 and R and R₁ are chosen from various organic and epoxidized substitutions. Further disclosed is an epoxy carboxylate of Formula (II): (R′—O)ₙ—R₁ (II) wherein n is an integer from 1 to 4, R₁ is chosen from various organic and epoxidized substitutions, and R′ is an epoxidized dicarboxylic acyl derived from a variety bioderived dicarboxylic acids.

### FIELD

Disclosed herein are epoxy biobased plasticizers and methods of preparation, compositions and use (e.g., rubber compositions for tires, treads, etc.) thereof.

### BACKGROUND

Tires are indispensable for both the automotive industry and overall mobility. Recently, the sustainability and environmental impact of tire materials have been questioned. Conventional tires are a typically formed of materials having a composite structure with components made from elastomers such as natural rubber (“NR”) and styrene-butadiene-rubber (“SBR”) in combination with various additives. Among the components, synthetic polymers, rubber process oils, and carbon blacks are non-renewable, and petroleum resourced. The rubber compound is usually reinforced with carbon black and may further include fatty acid additives (cure aid) and vulcanization aids. Synthetic rubber produced in this manner exhibits high abrasion resistance, good wet grip, and low rolling resistance.

However, of the automobile industry's share of 40% global pollution, tires contribute to a hefty 20-30%. Tire manufacturing also contributes to carbon dioxide emissions and the depletion of petrochemical resources. Several studies have shown a ubiquitous presence of the much-discussed tire and road wear particles (“TRWP”) in the air, water, and soil. Further, several degradation studies of tire particles show how tire leachates from the additives can be present in the environment after prolonged exposure. Moreover, TRWP is shown to have environmental implications like human health risk on the liver and kidneys and respiratory irritation, and acute toxicity on aquatic life.

There is an urgent need for sustainable tire formulations that incorporate more environmentally friendly materials. For example, there is a need for a rubber material for producing tire treads the major components of which are derived from natural sustainable resources. The material may suitably exhibit high tensile strength and good elongation at break values while maintaining desirable properties of a tire such as wet traction, low rolling resistance, good processability and abrasion resistance.

### BRIEF SUMMARY

Disclosed herein according to various embodiments are bio-based plasticizers, comprising: at least one epoxy soyate of Formula (I):

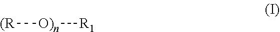

wherein:
   n is an integer from 1 to 4,
   R is chosen from C₁-C₃₆ alkyl, C₁-C₃₆ alkene, C₅-C₁₂ cycloalkyl, C₇-C₉ aralkyl, C₁-C₃₆ alkoxy, epoxidized linoleoyl, epoxidized ricinoleoyl, epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-epoxidized myristoyl, or non-epoxidized margaroyl, wherein each of the alkyl, alkene, cycloalkyl, aralkyl, or alkoxy is straight, branched, unsubstituted, or substituted with a straight or branched C₁-C₃₆ alkyl, halogen, nitrogen or sulfur, and
   R₁ is chosen from C₁-C₃₆ alkyl, C₁-C₃₆ alkene, C₂-C₁₂ alkylene, C₆-C₁₀ cycloalkylene, C₆-C₁₀ arylene, C₈-C₁₀ alkylenearylenealkylene, C₅-C₁₂ cycloalkyl, C₇-C₉ aralkyl, C₁-C₃₆ alkoxy, epoxidized linoleoyl, epoxidized ricinoleoyl epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-epoxidized myristoyl, or non-epoxidized margaroyl, wherein each of the alkyl, alkene, alkylene, cyhcloalkylene, arylene, alkylenearylenealkylene, cycloalkyl, aralkyl, or alkoxy is straight, branched, unsubstituted, or substituted with a straight or branched C₁-C₃₆ alkyl, halogen, nitrogen or sulfur; and/or
at least one epoxy carboxylate of Formula (II):

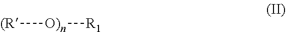

wherein:
   n is an integer from 1 to 4,
   R₁ is defined as above, and
   R′is an epoxidized dicarboxylic acyl derived from one of the following dicarboxylic acids:

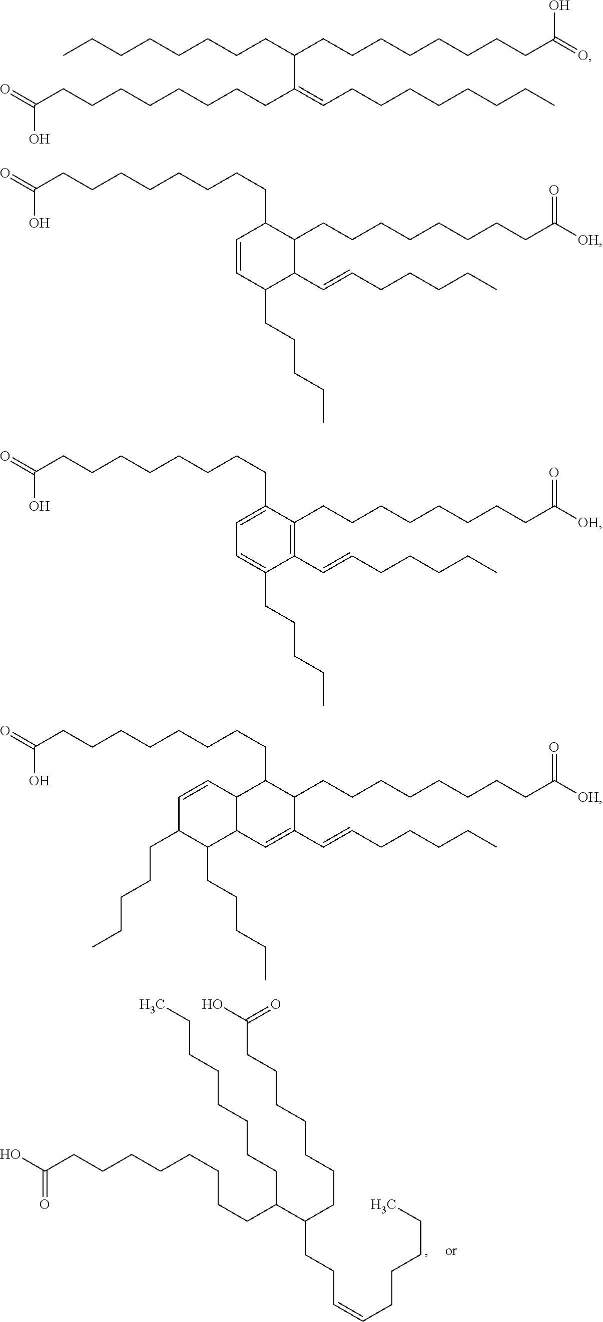

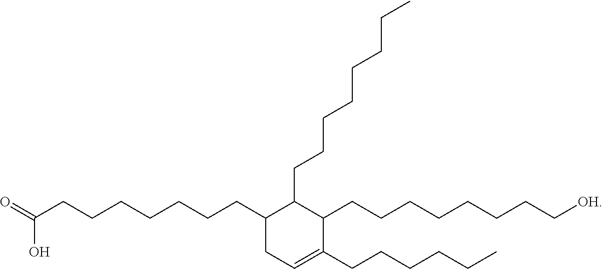 [SMILES: CCCCCCCCC1C(CCCCCCCC(=O)O)CC=C(CCCCCC)C1CCCCCCCCO]

An epoxy soyate or epoxy biobased carboxylate derived from a sugar of Formula (VII), (VIII) or (IX), comprising:

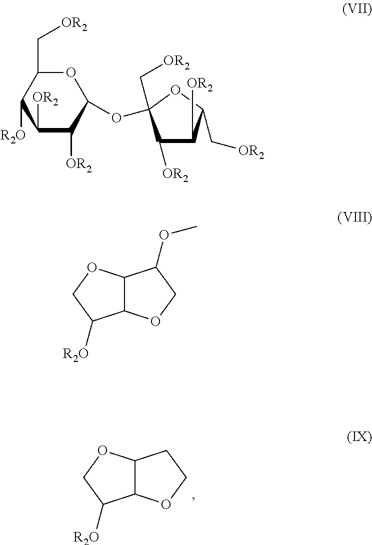

wherein each R₂ is independently chosen from epoxidized linoleoyl, epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-epoxidized myristoyl, non-epoxidized margaroyl, or epoxidized dicarboxylic acyl, derived one or more dicarboxylic acids.

An epoxy soyate or epoxy biobased carboxylate derived from a furan of Formula (X)-(XIII):

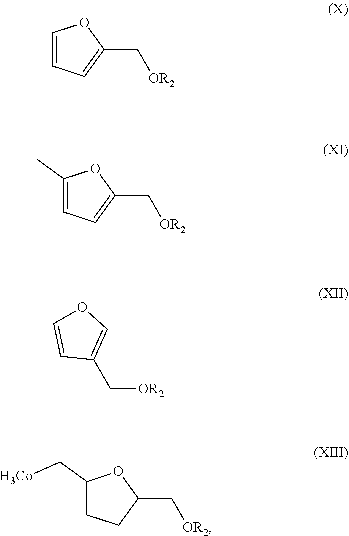

wherein each R₂ is independently chosen from epoxidized linoleoyl, epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-epoxidized myristoyl, non-epoxidized margaroyl, or epoxidized dicarboxylic acyl, derived one or more dicarboxylic acids.

An epoxy soyate or epoxy biobased carboxylate derived from a pyran of Formula (XIV)-(XIX):

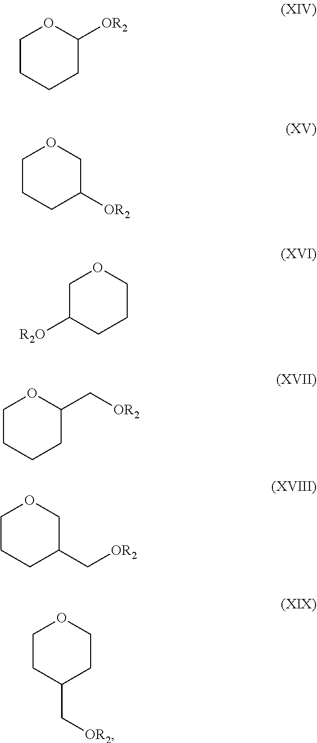

wherein each R₂ is independently chosen from epoxidized linoleoyl, epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-epoxidized myristoyl, non-epoxidized margaroyl, or epoxidized dicarboxylic acyl, derived one or more dicarboxylic acids.

An epoxy soyate or epoxy biobased carboxylate derived from a biobased dimer diol precursor of Formula (XX):

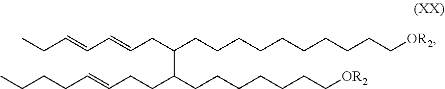

wherein each R₂ is independently chosen from an epoxidized linoleoyl, epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-epoxidized myristoyl, non-epoxidized margaroyl, or epoxidized dicarboxylic acyl derived from one of the following dicarboxylic acids:

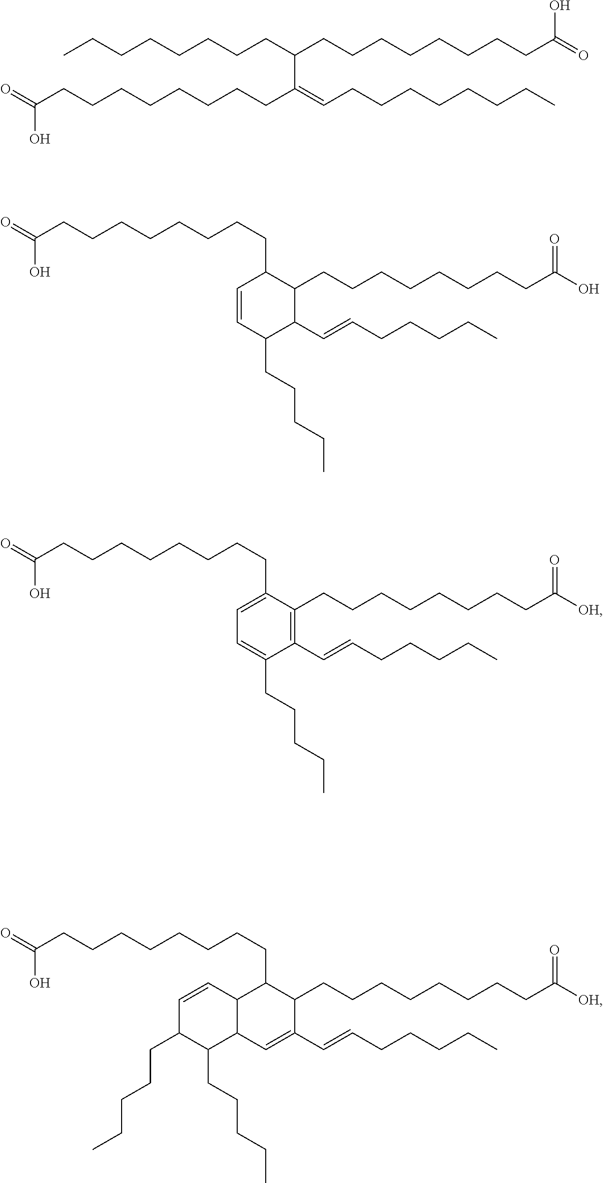 [SMILES: CCCCCCCC/C=C(/CCCCCCCCC(=O)O)C(CCCCCCCC)CCCCCCCCC(=O)O ; CCCCC/C=C/C1C(CCCCC)C=CC(CCCCCCCCC(=O)O)C1CCCCCCCCC(=O)O ; CCCCC/C=C/c1c(CCCCC)ccc(CCCCCCCCC(=O)O)c1CCCCCCCCC(=O)O ; CCCCC/C=C/C1=CC2C(C=CC(CCCCC)C2CCCCC)C(CCCCCCCCC(=O)O)C1CCCCCCCCC(=O)O]

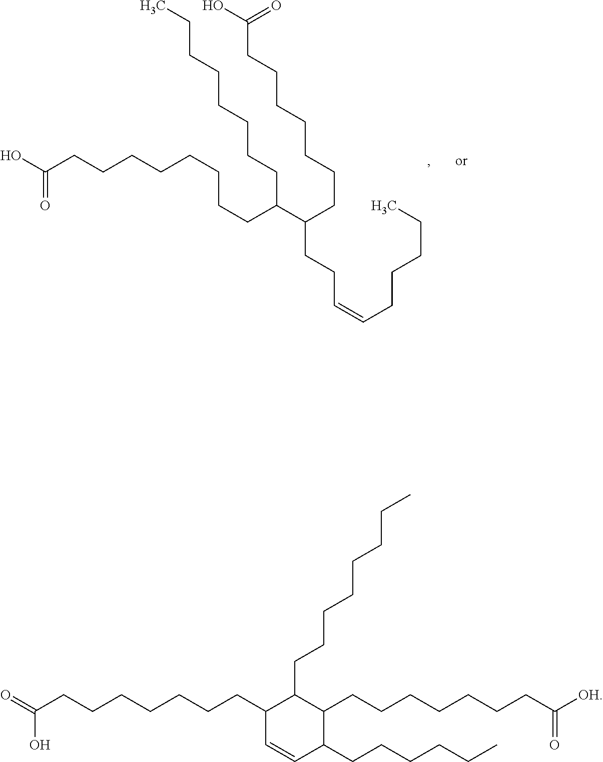 [SMILES: CCCCC/C=C\CCC(CCCCCCCC(=O)O)C(CCCCCCCC)CCCCCCCCC(=O)O ; CCCCCCCCC1C(CCCCCCCC(=O)O)C=CC(CCCCCC)C1CCCCCCCC(=O)O]

A bio-based plasticizer, comprising:
a plurality of epoxy soyates of Formula (XXI):

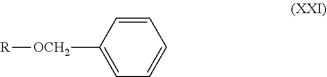

wherein each of the plurality of epoxy soyates independently comprises an R chosen from an epoxidized alpha-linolenoyl, epoxidized linoleoyl, epoxidized oleoyl, epoxidized ricinoleoyl, and/or epoxidized palmitoyl.

A bio-based plasticizer, comprising:
a plurality of epoxy carboxylates of Formula (XXII):

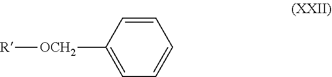

wherein each of the plurality of epoxy carboxylates independently comprises an R′ chosen from an epoxidized dicarboxylic acyl derived from one of the following dicarboxylic acids:

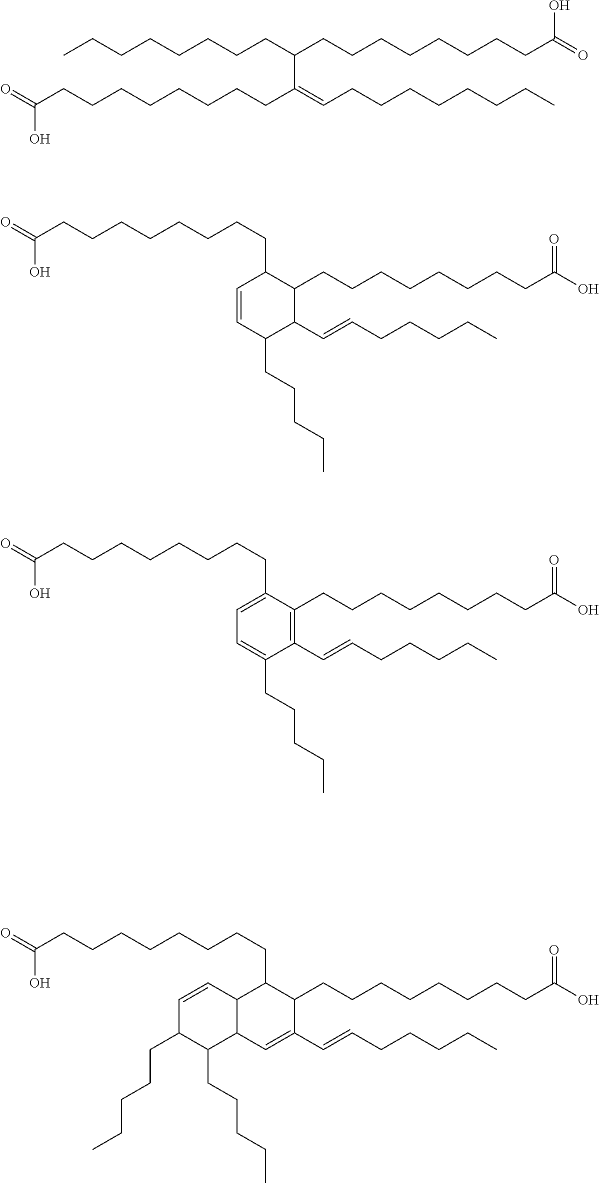 [SMILES: CCCCCCCC/C=C(/CCCCCCCCC(=O)O)C(CCCCCCCC)CCCCCCCCC(=O)O ; CCCCC/C=C/C1C(CCCCC)C=CC(CCCCCCCCC(=O)O)C1CCCCCCCCC(=O)O ; CCCCC/C=C/c1c(CCCCC)ccc(CCCCCCCCC(=O)O)c1CCCCCCCCC(=O)O ; CCCCC/C=C/C1=CC2C(C=CC(CCCCC)C2CCCCC)C(CCCCCCCCC(=O)O)C1CCCCCCCCC(=O)O]

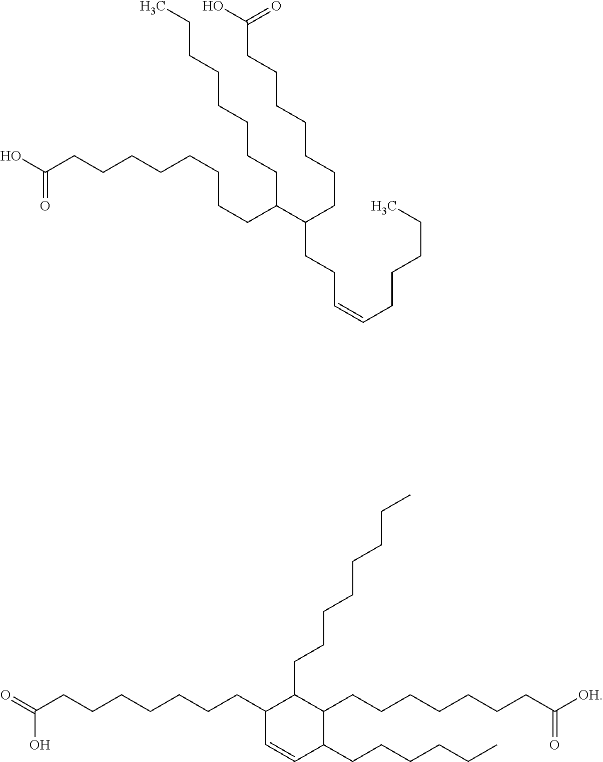 [SMILES: CCCCC/C=C\CCC(CCCCCCCC(=O)O)C(CCCCCCCC)CCCCCCCCC(=O)O ; CCCCCCCCC1C(CCCCCCCC(=O)O)C=CC(CCCCCC)C1CCCCCCCC(=O)O]

A bio-based plasticizer, comprising:
a plurality of epoxy soyates of Formula (XXIII):

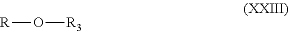

wherein each of the plurality of epoxy soyates independently comprises:
   an R chosen from epoxidized alpha-linolenoyl, epoxidized linoleoyl, or epoxidized oleoyl, and
   an R₃ chosen from 2-chlorobenzyl, 3-chlorobenzyl, 4-chlorobenzyl, 2-bromobenzyl, 3-bromobenzyl, 4-bromobenzyl, 2-methoxybenzyl, 3-methoxybenzyl, or 4-methoxybenzyl.

A bio-based plasticizer, comprising:
a plurality of epoxy carboxylates of Formula (XXIV):

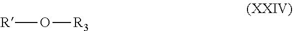

wherein each of the plurality of epoxy carboxylates independently comprises:
   an R′ chosen from an epoxidized dicarboxylic acyl derived from the following dicarboxylic acids:

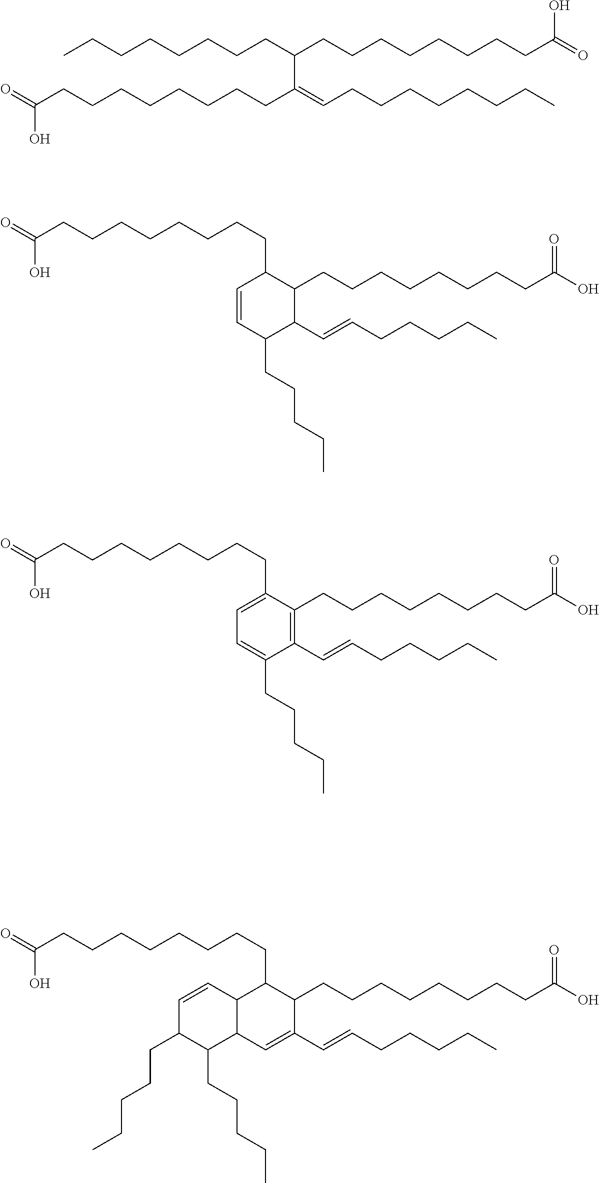 [SMILES: CCCCCCCC/C=C(/CCCCCCCCC(=O)O)C(CCCCCCCC)CCCCCCCCC(=O)O ; CCCCC/C=C/C1C(CCCCC)C=CC(CCCCCCCCC(=O)O)C1CCCCCCCCC(=O)O ; CCCCC/C=C/c1c(CCCCC)ccc(CCCCCCCCC(=O)O)c1CCCCCCCCC(=O)O ; CCCCC/C=C/C1=CC2C(C=CC(CCCCC)C2CCCCC)C(CCCCCCCCC(=O)O)C1CCCCCCCCC(=O)O]

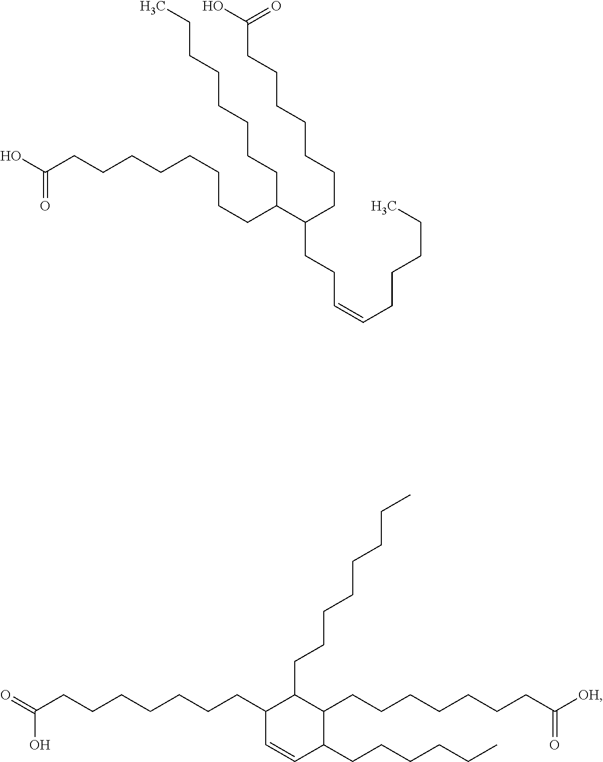 [SMILES: CCCCC/C=C\CCC(CCCCCCCC(=O)O)C(CCCCCCCC)CCCCCCCCC(=O)O ; CCCCCCCCC1C(CCCCCCCC(=O)O)C=CC(CCCCCC)C1CCCCCCCC(=O)O]

and
an R₃ chosen from benzyl, 2-chlorobenzyl, 3-chlorobenzyl, 4-chlorobenzyl, 2-bromobenzyl, 3-bromobenzyl, 4-bromobenzyl, 2-methoxybenzyl, 3-methoxybenzyl, 4-methoxybenzyl, C₁-C₃₆ alkyl, or C₅-C₁₂ cycloalkyl wherein the benzyl, alkyl and cycloalkyl may be unsubstituted or substituted with one or more halogen, nitrogen, or sulfur.

A tire, comprising: a plasticizer according to embodiments herein.

A rubber, comprising: a plasticizer according to embodiments herein.

A rubber component, comprising: a plasticizer according to embodiments herein.

### Definitions

Reference throughout this specification to, for example, “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “In one or more embodiments” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “an adsorption vessel” includes a single adsorption vessel as well as more than one adsorption vessel.

As used herein, the term “about” in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Unless otherwise indicated, all parts and percentages are by weight. Weight percent (wt. %), if not otherwise indicated, is based on an entire composition free of any volatiles, that is, based on dry solids content.

The term “phr” as used herein means parts by weight per 100 parts of component (e.g., elastomer).

The term “plasticizer” as used herein refers to a compound having any of the chemical formulas disclosed herein, or a composition containing one or more of the compounds and optionally additional components.

### DETAILED DESCRIPTION

Embodiments of the disclosure are described more fully hereinafter with reference to the accompanying drawings, which form a part hereof, and which show, by way of illustration, example features. The features can, however, be embodied in many different forms and should not be construed as limited to the combinations set forth herein; rather, these combinations are provided so that this disclosure will be thorough and complete and will fully convey the scope. The following detailed description is, therefore, not to be taken in a limiting sense.

Disclosed herein according to various embodiments are rubber compositions for tires, more specifically rubber compositions for a tread. Described, according to various embodiments, are soy and other biobased plasticizers, namely epoxy soyates, for the manufacture of tire treads, method of preparation, compositions (e.g., tires, rubber, rubber components) and methods of use thereof (e.g., to manufacture tire tread made from the plasticizers).

In one or more embodiments, disclosed are rubber compositions, which can be used, for example, as treads for winter tires. Such winter tires are suitable for rolling over ground surfaces, for example, covered with ice or black ice, without needing studs.

According to various embodiments, further disclosed are treads for winter tires, for example, suitable to roll during “melting ice” conditions within a temperature range of about −5° C. to about 0° C. Within such a range, the pressure of the tires while the vehicle is in operation causes surface melting of the ice, which is covered with a thin film of water that impacts the grip of tires.

In at least one embodiment, disclosed is the use of epoxy soyates and other epoxy biobased esters as plasticizers in other rubber-based formulations including mats, conveyor belts, drive belts and rollers, hoses, food processing equipment, sports equipment, gloves rubber flooring, shoes. wet suits, clothing, playground equipment, industrial rollers and seals.

In various embodiments, described is a rubber composition usable as tread for a winter tire and which comprises at least a diene elastomer, more than 30 phr of novel soy based liquid plasticizers, about 50 phr to about 150 phr of a reinforcing filler, and about 5 to about 40 phr of magnesium sulphate microparticles, or any individual value or sub-range within these ranges.

Further described herein according to embodiments is a rubber composition, which is capable of generating an effective surface micro-roughness by virtue of specific water-soluble microparticles and which makes it possible to improve the grip on ice of the treads and tires comprising them under melting ice conditions without being disadvantageous to the properties of reinforcement and hysteresis. Such a rubber composition can be used in the manufacture of treads for winter tires, whether the treads are intended for new tires or for the retreading of worn tires.

According to further embodiments are treads and tires formed of a rubber composition as described herein. Such tires are suitable to equip passenger motor vehicles, including four-wheel drive (4×4) vehicles, sport utility vehicles (“SUV”), two-wheel vehicles (e.g., motorcycles), and industrial vehicles such as vans, heavy-duty vehicles (e.g., underground, bus or heavy road transport vehicles such as lorries, tractors, trailers, etc.), and off-road vehicles such as agricultural vehicles and earthmoving equipment.

Further described in one or more embodiments is the use of epoxy soyates as plasticizers in other rubber-based formulations. Such formulations are suitable as materials for mats, conveyor belts, drive belts and rollers, hoses, food processing equipment, sports equipment, gloves, rubber flooring, shoes, wet suits, clothing, playground equipment, industrial rollers and/or seals.

In various embodiments, rubber compositions described herein are based on at least a diene elastomer, a plasticizing system, a reinforcing filler and magnesium sulphate microparticles, which components are described in detail below.

### Diene Elastomers

It should be remembered that diene elastomer or rubber should be understood as meaning an elastomer resulting at least in part (i.e., a homopolymer or a copolymer) from diene monomers (monomers carrying two carbon-carbon double bonds which may or may not be conjugated). Diene elastomers can be classified in a known way into two categories: those “essentially unsaturated” and those “essentially saturated.” Butyl rubbers, such as, for example copolymers of dienes and of C-olefins of EPDM type, come within the category of “essentially saturated” diene elastomers, having a content of units of diene origin which is low or very low, always less than 15% (mol %). In contrast, “essentially unsaturated” diene elastomers include a diene elastomer resulting at least in part from conjugated diene monomers, having a content of units of diene origin (conjugated dienes) that is greater than 15% (mol %). In the category of “essentially unsaturated” diene elastomers, “highly unsaturated” diene elastomer is understood to mean, for example, a diene elastomer having a content of units of diene origin (conjugated dienes) which is greater than 50%. In some embodiments, at least one diene elastomer is used. Suitable diene elastomers include the highly unsaturated type, for example, a diene elastomer chosen from polybutadienes (“BR”), synthetic polyisoprenes (“IR”), natural rubber, butadiene copolymers, isoprene copolymers (other than IIR) and mixtures of these elastomers. Such copolymers may be chosen from butadiene/styrene copolymers, isoprene/butadiene copolymers (“BIR”), isoprene/styrene copolymers (“SIR”), isoprene/butadiene/styrene copolymers (“SBIR”) and mixtures of such copolymers. The elastomers can, for example, be block, random, sequential or micro sequential elastomers and can be prepared in dispersion or in solution; they can be coupled and/or star branched or also functionalized with a coupling and/or star branching or functionalization agent. For coupling with car bon black, mention may be made, for example, of functional groups comprising a C Sn bond or of aminated functional groups, such as benzophenone, for example; for coupling with a reinforcing inorganic filler, such as silica, mention may be made, for example, of silanol functional groups or polysiloxane functional groups having a silanol end, of alkoxysilane groups, of carboxyl groups, or of polyether groups. Mention may also be made, as other examples of such functionalized elastomers, of elastomers (such as SBR, BR, NR or IR) of the epoxidized type.

Suitable elastomers include, in embodiments, polybutadienes, for example, those having a content of 1.2-units of about 4% to about 80% or those having a content of cis-1,4-units of greater than about 80%, polyisoprenes, butadiene/styrene copolymers, for example, those having a styrene content of about 5% to about 50% by weight, about 20% to about 40%, a content of 1.2-bonds of the butadiene part of about 4% to about 65%, and a content of trans-1,4-bonds of about 20% to about 80%, butadiene/isoprene copolymers, for example, those having an isoprene content of about 5% to about 90% by weight, and a glass transition temperature (“Tg-measured according to ASTM D 3418-82) of −40° C. to −80° C., or isoprene/styrene copolymers, for example, those having a styrene content of about 5% to about 50% by weight and a Tg of about −25° C. to about −50° C., or any individual value or sub-range within these ranges.

In the case of butadiene/styrene/isoprene copolymers, those having a styrene content of about 5% to about 50% by weight, or about 10% to about 40%, an isoprene content of about 15% to about 60% by weight, or about 20% to about 50%, a butadiene content of about 5% to about 50% by weight or about 20% to about 40%, a content of 1.2-units of the butadiene part of about 4% to about 85%, a content of trans-1,4-units of the butadiene part of about 6% to about 80%, a content of 1.2-plus 3,4-units of the isoprene part of about 5% to about 70% and a content of trans-1,4-units of the isoprene part of about 10% to about 50%, and/or any butadiene/styrene/isoprene copolymer having a Tg of about −20° C. to about −70° C., or any individual value or sub-range within these ranges, are suitable.

According to one or more embodiments, the diene elastomer is chosen from the group consisting of natural rubber, synthetic polyisoprenes, polybutadienes having a content of cis-1,4 bonds of greater than about 90%, or any individual value or sub-range within this range, butadiene/styrene copolymers and the mixtures of these elastomers.

According to at least one embodiment, the diene elastomer used is greater than about 50 phr, or any individual value or sub-range within this range, natural rubber or a synthetic polyisoprene. In some embodiments, the said natural rubber or synthetic polyisoprene is then used as a blend with a polybutadiene having a content of cis-1,4 bonds which is greater than about 90%.

In one or more embodiments, the diene elastomer used is present in an amount of greater than about 50 phr, and the composition may further include a polybutadiene having a content of cis-1.4 bonds of greater than about 90%. In some embodiments, said polybutadiene is then used as a blend with natural rubber or a synthetic polyisoprene.

In various embodiments, the diene elastomer used is a binary blend (mixture) of NR (or IR) and of BR, or a ternary blend of NR (or IR), BR and SBR. In some embodiments of such blends, the composition comprises about 25 phr to about 75 phr of NR (or IR) and about 75 phr to about 25 phr of BR, with which may or may not be associated a third elastomer (ternary blend) at a content of less than about 30 phr, or of less than about 20 phr, or any individual value or sub-range within these ranges. This third elastomer may be an SBR elastomer, for example, a solution SBR (“SSBR). In one or more embodiments of such a blend, the composition comprises from about 35 phr to about 65 phr of NR (or IR) and from about 65 phr to about 35 phr of BR, or any individual value or sub-range within these ranges. In some embodiments, the BR used is has a content of cis-1.4 bonds of greater than about 90%, greater than about 95%, or any individual value or sub-range within these ranges.

Synthetic elastomers other than diene elastomers, indeed even polymers other than elastomers, for example thermoplastic polymers, might be combined, in a minor amount, with the diene elastomers of the compositions according to embodiments herein.

In some embodiments, any rubber that can be crosslinked by a sulfur cure can be used in the compositions. Sulfur cure describes a vulcanization process typical of making rubber. In other embodiments, rubber capable of being cured by a peroxide crosslinking mechanism may be used. The rubbers may be natural rubber or synthetic rubbers. Examples of synthetic rubbers include without limitation, synthetic polyisoprenes, polybutadienes, acrylonitrile butadiene rubber, styrene acrylonitrile butadiene rubber, poly chloroprene rubber, styrene-butadiene copolymer rubber, isoprene isobutylene copolymer rubber and its halogenated derivatives, ethylene-propylene-diene copolymer rubbers Such as ethylene-propylene-cyclopentadiene terpolymer, ethylene-propylene-5-ethylidene-norbornene terpolymer, and ethylene-propylene-1,4-hexadiene terpolymer, butadiene-propylene copolymer rubber, butadiene-ethylene copolymer rubber, butadiene-isoprene copolymer, polypentenamer, millable urethanes and their mixtures. In one aspect, such compounds are characterized by repeating olefinic unsaturation in the backbone of the polymer, which may arise, for example, from the presence of butadiene or isoprene monomers in the polymer structure.

### Reinforcing Filler

Use may be made of any type of reinforcing filler known for its capabilities of reinforcing a rubber composition which can be used for the manufacture of tires, for example an organic filler, such as carbon black, or a reinforcing inorganic filler, such as silica, with which a coupling agent is combined in a known way.

Such a reinforcing filler typically consists of nanoparticles, the mean size (by weight) of which is less than about 500 nm, or about 20 nm to about 200 nm, or about 20 to about 150 nm, or any individual value or sub-range within these ranges.

All carbon blacks, for example, blacks of the HAF, ISAF or SAF type, conventionally used in treads for tires (“tire-grade” blacks) are suitable as carbon blacks.

Mention will more particularly be made, among the latter, of the reinforcing

Carbon blacks of the 100, 200 or 300 series (ASTM grades), such as, for example, the N115, N134, N234, N326, N330, N339, N347 or N375 blacks. The carbon blacks might, for example, be already incorporated in the isoprene elastomer in the form of a master batch. Mention may be made, as examples of organic fillers other than carbon blacks, of the functionalized polyvinyl organic fillers.

The term “reinforcing inorganic filler should be under stood here as meaning any inorganic or mineral filler, whatever its color and its origin (natural or synthetic), also known as “white filler” or sometimes “clear filler” in contrast to carbon black, capable of reinforcing by itself, without means other than an intermediate coupling agent, a rubber composition intended for the manufacture of tires, in other words capable of replacing, in its reinforcing role, a conventional tire-grade carbon black; such a filler is generally characterized, in a known way, by the presence of hydroxyl (—OH) groups at its surface.

Mineral fillers of the siliceous type, for example, silica (SiO₂), or of the aluminous type, for example, alumina (Al₂O₃), are suitable for example, as reinforcing inorganic fillers. The silica used can be any reinforcing silica known to a person skilled in the art, for example, any precipitated or pyrogenic silica exhibiting a BET surface and a CTAB specific surface both of less than about 450 m/g, about 30 m/g to about 400 m/g, about 60 m/g to about 300 m/g, or any individual value or sub-range within these ranges. Mention will be made, as highly dispersible (“HD precipitated silicas’), for example, of the Ultrasil 7000 and Ultrasil 7005 silicas from Degussa, the Zeosil 1165 MP, 1135 MP and 1115 MPsilicas from Rhodia, the Hi-Sil EZ150G silica from PPG, the Zeopol 8715,8745 and 8755 silicas from Huber. Mention may be made, as examples of reinforcing aluminas, of the “Baikalox A125′ or “Baikalox CR 125 aluminas from Baikowski, the “APA-100RDX alumina from Condea, the “Aluminoxid Calumina from Degussa or the “AKP-G015” alumina from Sumitomo Chemicals.

In at least one embodiment, the content of total reinforcing filler (carbon black and/or reinforcing inorganic filler) is about 60 phr to about 120 phr, about 70 phr to about 100 phr, or any individual value or sub-range within these ranges.

According to at least one embodiment, the reinforcing filler comprises predominantly carbon black; in such a case, the carbon black is present in an amount of greater than about 60 phr, or any individual value or sub-range within this range, optionally in combination with a reinforcing inorganic filler, such as silica, in a minor amount.

According to another specific embodiment, the reinforcing filler comprises predominantly an inorganic filler, for example, silica; in Such a case, the inorganic filler, for example, silica, is present in an amount of greater than about 70 phr, or any individual value or sub-range within this range, optionally in combination with carbon black in a minor amount; the carbon black, when it is present, is used in an amount of less than about 20 phr, less than about 10 phr, or about 0.1 to about 10 phr, or any individual value or sub-range within these ranges. Separate from the search for optimized grip on melting ice, a predominant use of a reinforcing inorganic filler, such as silica, is also advantageous from the viewpoint of the grip on a wet or Snowy ground Surface.

According to one or more embodiments, the reinforcing filler comprises a blend of carbon black and of reinforcing inorganic filler Such as silica, in similar amounts; in such a case, the content of inorganic filler, for example, silica, and the content of carbon black are each about 25 phr to about 75 phr, about 30 phr to about 50 phr, or any individual value or sub-range within these ranges.

In order to couple the reinforcing inorganic filler to the diene elastomer, use may be made of a bifunctional coupling agent (or bonding agent) intended to provide a satisfactory connection, of chemical and/or physical nature, between the inorganic filler (surface of its particles) and the diene elastomer. Use is made, for example, of bifunctional organosilanes or polyorganosiloxanes.

Use is made for example, of silane polysulphides, referred to as “symmetrical” or “asymmetrical” depending on their specific structure. “Symmetrical” silane polysulphides corresponding to the following general Formula (A):

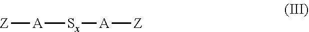

wherein:
x is an integer from 2 to 8, or 2 to 5;
A is a divalent hydrocarbon radical such as C₁-C₁₈ alkylene groups or C₆-C₁₂ arylene groups, C₁-C₁₀ or C₁-C₄ alkylenes, or propylene;
Z corresponds to one of the formulae (IV), (V) or (VI) below:

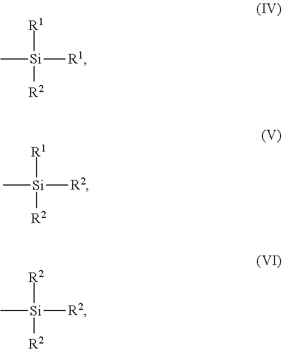

wherein:
   the R¹ radicals, which may be unsubstituted or substituted and identical to or different from one another, represent a C₁-C₁₈ alkyl, C₅-C₁₈ cycloalkyl or C₆-C₁₈ aryl group (e.g., C₁-C₆ alkyl, cyclohexyl or phenyl groups such as C₁-C₄ alkyl groups, and methyl and/or ethyl),
   the R² radicals, which may be unsubstituted or substituted and identical to or different from one another, represent a C₁-C₁₈ alkoxy or C₅-C₁₈ cycloalkoxy group (e.g., a group chosen from C₁-C₈ alkoxyls and C₅-C₈ cycloalkoxy, or a group chosen from C₁-C₄ alkoxyls, such as, methoxy and ethoxy), are suitable without limitation.

Suitable exemplary silane polysulphides include bis(3-trimethoxysilylpropyl) or bis(3-triethoxysilylpropyl)polysulphides. Use is made, for example, among compounds of bis(3-triethoxysilylpropyl)tetra sulfide (“TESPT”), or bis(triethoxysilylpropyl)disulfide (“TESPD”). Mention will also be made of bis(mono(C₁-C₄)alkoxy di(C₁-C₄)alkylsilylpropyl)polysulfides (e.g., disulfides, trisulfides or tetrasulfides), for example, bis(monoethoxydimethylsilylpropyl) tetrasulfide.

Suitable coupling agents other than alkoxysilane polysulfide, include, but are not limited to, bifunctional POSS (polyorganosiloxanes) or of hydroxysilane polysulphides (R²═OH in the above Formulas (IV)-(VI)), or of silanes or POSs carrying azodicarbonyl functional groups.

Rubber compositions according to one or more embodiments herein include a coupling agent in an amount of about 2 phr to about 12 phr, about 3 phr to about 8 phr, or any individual value or sub-range within these ranges.

A person skilled in the art will understand that a reinforcing filler of another nature, for example, organic nature, might be used as filler equivalent to the reinforcing inorganic filler described in the present section, provided that this reinforcing filler is covered with an inorganic layer, such as silica, or else comprises, at its surface, functional sites, for example, hydroxyls, requiring the use of a coupling agent in order to form the connection between the filler and the elastomer.

### Magnesium Sulphate Microparticles

Rubber compositions according to one or more embodiments herein include magnesium sulphate microparticles in the amount of about 5 phr to about 40 phr, or any individual value or sub-range within this range.

Microparticles is understood to mean, by definition and in general, particles of micrometric size, that is to say for which the mean size or median size (both expressed by weight) are about 1 μto about 1 mm, about 2 μm to about 800 μm, or any individual value or sub-range within these ranges.

Below the minima indicated above, there is a risk that the targeted technical effect (namely the creation of a suitable micro-roughness) will be inadequate whereas, above the maxima indicated, various disadvantages emerge, for example, when the rubber composition is used as tread: apart from a possible aesthetic loss (particles too visible on the surface of the tread) and a risk of loss of cohesion during rolling of relatively large elements of the tread pattern, it being found that the grip performance on melting ice may be damaged.

For all these reasons, the microparticles suitably have a median size of about 2 μm to about 500 μm, about 5 to about 200 μm, or any individual value or sub-range within these ranges. This size range appears to correspond to an optimized compromise between, on the one hand, a desired surface roughness and, on the other hand, good contact between the rubber composition and the ice.

Moreover, for identical reasons to those set out above, the content of microparticles is about 5 phr to about 40 phr, about 10 phr to about 35 phr, or any individual value or sub-range within these ranges.

Various known methods are applicable for the analysis of the particle size and the calculation of the median size of the microparticles (or median diameter for microparticles assumed to be substantially spherical), for example by laser diffraction (see, for example Standard ISO-8130-13 or Standard HS K5600-9-3).

Use may also be made of an analysis of the particle size by mechanical sieving; the operation consists of sieving a defined amount of sample (e.g., about 200 g) on a vibrating table for 30 min with different sieve diameters (for example, according to a progressive ratio equal to 1.26, with meshes of 1000, 800, 630, 500, 400, . . . 100, 80, and 63 μm); the oversize collected in each sieve is weighed on a precision balance; the % of oversize for each mesh diameter with respect to the total weight of product is deduced therefrom; the median size (or median diameter) or mean size (or mean diameter) is finally calculated in a known way from the histogram of the particle size distribution.

### Plasticizing System

The rubber composition according to one or more embodiments herein includes at least 30 phr of a plasticizing agent which is liquid (at 23° C.), the role of which is to soften the matrix by diluting the elastomer and the reinforcing filler; its Tg is by definition less than about −20° C., less than about −40° C., or any individual value or sub-range within these ranges.

Any extending oil, whether of aromatic or non-aromatic nature, any liquid plasticizing agent known for its plasticizing properties with regard to diene elastomers, can be used. At ambient temperature (23° C.), these plasticizers or these oils, which are more or less viscous, are liquids (that is to say, as a reminder, substances that have the ability to eventually take on the shape of their container), as opposed, for example, to plasticizing hydrocarbon resins which are by nature solid at ambient temperature.

Liquid plasticizers chosen from the group consisting of naphthenic oils (low or high viscosity, for example, hydrogenated or otherwise), paraffinic oils, MES (Medium Extracted Solvates) oils, TDAE oils (Treated Distillate Aromatic Extracts), mineral oils, plant oils, ether plasticizers, ester plasticizers, phosphate plasticizers, sulphonate plasticizers and the mixtures of these compounds are particularly suitable.

Mention may be made, as phosphate plasticizers for example, of those that contain about 12 to about 30 carbon atoms,, or any individual value or sub-range within this range, for example trioctyl phosphate. As examples of ester plasticizers, mention may especially be made of the compounds chosen from the group consisting of trimellitates, pyromellitates, phthalates, 1,2-cyclohexane dicarboxylates, adipates, azelates, sebacates, triesters of glycerol, and mixtures of these compounds. Among the above triesters, mention may be made of glycerol triesters, for example, composed predominantly (for more than about 50% by weight, more than about 80% by weight, or any individual value or sub-range within these ranges) of an unsaturated C₁₈ fatty acid, that is to say an unsaturated fatty acid chosen from oleic acid, linoleic acid, linolenic acid and mixtures of these acids. In some embodiments, whether of synthetic origin or natural origin (in the case, for example, of sunflower or rapeseed vegetable oils), the fatty acid used is present in an amount of greater than about 50% by weight, or greater than about 80% by weight, or any individual value or sub-range within these ranges, of oleic acid. Such triesters (trioleates) comprising a high content of oleic acid are well known may be used as plasticizing agents in treads for tires.

In some embodiments, an epoxidized fatty acid ester, such as epoxidized alkyl soyate, is useful in this composition. Any plasticizer containing fatty acids derived from vegetable oils and which have been substantially fully esterified with an alcohol (mono alcohol or polyol) and having unsaturated bonds that are substantially fully epoxidized is a candidate for use as a plasticizer herein. Moreover, it is helpful if the fatty acids are added substantially randomly to one or more hydroxyl sites on the alcohol.

Any epoxidized alkyl soyate is a particularly suitable candidate for use in compositions according to embodiments herein. It is understood that “soyate” is a carboxylate moiety which refers to any naturally occurring or subsequently refined mixture of fatty acids and their esters, where the fatty acids include stearic acid, oleic acid, linoleic acid, linolenic acid, and the like. Epoxidation of such fatty acid esters typically generates an epoxy group, also called a glycidyl group or oxirane ring, replacing a double bond in the fatty acid backbone.

U.S. Pat. No. 6,797,753 (Benecke et al.), the disclosure of which is incorporated by reference in its entirety, teaches the value of these epoxidized esters from fatty acids, including manufacture of a number of epoxidized esters from fatty acids, including epoxidized propylene glycol disoyate and epoxidized methyl soyate (EMS).

Benecke et al. teach the use of any vegetable or plant fatty acid glyceride that is significantly unsaturated. “Significantly unsaturated” means that the vegetable oil typically has more than about 80% unsaturated fatty adds. In some embodiments, the unsaturation should be about 84% or higher. Typically, the oil has an iodine value, which is a measurement of the amount of double bonds in the fatty acids of the oil that is about 100 and higher.

Iodine Value (IV) is a measure of unsaturation and a good indicator of the extent of possible epoxidation. Examples of acceptable oils as sources for fatty acid derivatives and their respective IVs include: soybean oil (IV about 120-143), canola oil (IV about 100-115), corn oil (IV about 118-128), linseed oil (IV about 170-200), rapeseed oil (IV about 100-115), safflower oil (IV about 140-150), sunflower oil (IV about 125-140), tall oil (IV about 140-190), and tung oil (IV about 180) (and mixtures and derivatives thereof) all of which have an adequate number of unsaturated fatty acids (e.g., oleic, linolenic, linoleic) which are suitable for epoxidation.

Typically, the unsaturated fatty acids useful in the compositions described herein are selected from the random mix of unsaturated fatty acids present in the vegetable oil, the saturated fatty acids are likewise selected from the random mix of saturated fatty acids present in the vegetable oil. The identifying portions of saturated fatty acids present are termed saturated acyl groups that are derived from saturated fatty acids and are typified by palmitoyl, stearoyl, arachidoyl, behenoyl, myristoyl, and margaroyl.

Embodiments herein include the following soybean oil-derived plasticizers which are useful as primary plasticizers in plastics, such as polyvinyl chloride: (i) epoxidized pentaerythritol tetrasoyate; (ii) epoxidized propylene glycol disoyate; (iii) epoxidized ethylene glycol disoyate; (iv) epoxidized methyl soyate; (v) epoxidized sucrose octasoyate; and (vi) the epoxidized product of soybean oil interesterified with linseed oil (epoxidized interesterified soybean oil), for example, alkyl epoxy soyate such as benzyl epoxy soyate and 2-ethylhexyl epoxy soyate.

An epoxidized alkyl soyate is suitable for use in compositions described herein. It is understood that “soyate” is a carboxylate moiety which refers to any naturally occurring or subsequently refined mixture of fatty acids and their esters, where the fatty acids include stearic acid, oleic acid, linoleic acid, linolenic acid, and the like. Epoxidation of such fatty acid esters, such as methyl soyate, typically generates an epoxy group, also called a glycidyl group or oxirane ring, replacing a double bond in the fatty acid backbone. Non-limiting examples of epoxidized alkyl soyates include epoxidized methyl soyate, epoxidized ethyl soyate, epoxidized butyl soyate, epoxidized octyl soyate, epoxidized 2-ethyl hexyl soyate, benzyl epoxy soyate and combinations thereof.

In one or more embodiments, suitable vegetable oil-based plasticizers are made by a a method that comprises (i) creating ester linkages by attaching fatty acids derived from vegetable oils (e.g., oleic, linoleic, linolenic acid, and palmitoleic acid, etc.) to monoalcohols (monools) or polyalcohols (polyols) by means of direct esterification; and (ii) epoxidizing the esterified products (which contain saturated or unsaturated fatty acids) from step (i) to increase the polarity and increase the solubility parameter of these reaction products in the formulation. Presumably, increasing the polarity and solubility parameters increases the compatibility of the vegetable-oil based plasticizer in tire formulations. Alternatively, the first step of this broad method (direct esterification) may be substituted with the step of transesterification, wherein a monool or polyol reacts directly with the vegetable oil to produce the desired product plus glycerin, and wherein a monool or polyol reacts with the lower alkyl ester of vegetable oil acid to produce the desired product plus the lower alcohol. Typically, the saturated and unsaturated fatty acids are distributed randomly on each molecule of a polyol that is esterified with the fatty acids. This process also results in a random mix of esterified fatty acids.

An alternative broad embodiment includes the steps of (i) interesterification of one ester with another ester, or of a vegetable oil such as soybean oil with another vegetable oil such as linseed oil; and (ii) subsequent epoxidation of the product of the interesterification reaction. Interesterified oil may be further reacted with alcohols (monools and polyols) by transesterification of the interesterified product, followed by epoxidation of the trans esterified, interesterified product. The above-mentioned alternative may also be used here.

Modified vegetable-oil based plasticizers according to one or more embodiments are derived from soybean oil and include: (i) epoxidized pentaerythritol tetrasoyate; (ii) epoxidized propylene glycol disoyate; (iii) epoxidized ethylene glycol disoyate; (iv) epoxidized methyl soyate; (v) epoxidized 2-ethylhexyl soyate; (vi) epoxidized benzyl soyate (vii) epoxidized sucrose octasoyate; and (viii) the epoxidized product of soybean oil interesterified with linseed oil, and, for example, alkyl epoxy soyate such as benzyl epoxy soyate and 2-ethylhexyl epoxy soyate.

An object of various embodiments herein is to provide vegetable oil-based plasticizers which are useful as primary plasticizers, and which can completely replace petroleum-based compounds, such as DOP, as the primary plasticizers used in tire formulations.

The content of liquid plasticizer in compositions according to one or more embodiments herein is greater than 40 phr, or about 40 phr to about 100 phr, or any individual value or sub-range within these ranges.

### Additives

Rubber compositions according to one or more embodiments include one or more additives. Suitable additives include, but are not limited to, curing accelerators, antioxidants, fillers, fibers, and process aids such as plasticizers, protection agents such as antiozone waxes, chemical antiozonants, antioxidants, reinforcing resins, methylene acceptors (e.g., phenolic novolak resin) or methylene donors (e.g., HMT or H3M), a crosslinking system based either on sulfur or on donors of sulfur and/or peroxide and/or bismaleimides, vulcanization accelerators, vulcanization activators, or combinations of any two or more thereof. These compositions can also comprise coupling activators when a coupling agent is used, agents for covering the inorganic filler or more generally processing aids capable, in a known way, by virtue of an improvement in the dispersion of the filler in the rubber matrix and of a lowering of the viscosity of the compositions, of improving their property of processing in the raw state; these agents are, for example, hydrolysable silanes, such as alkylalkoxysilanes, polyols, polyethers, amines, or hydroxylated or hydrolysable polyorganosiloxanes.

A neutralizing agent that acts as a base addition may also be incorporated in compositions according to embodiments herein. In one or more embodiments, the neutralizing agent used is calcium stearate which may be added in an amount of 1 to 5 parts by weight. The neutralizing agent acts as a scorch safety agent during the vulcanization process. The acidity of the rubber compound causes the ring-opening of the epoxy group of ENR during the coagulation of the rubber. This causes the rubber compound to lose its ageing resistance. When the acidity of the rubber compound is neutralized, the rubber is able to coagulate efficiently and form stronger bonds and increase ageing resistance.

In various embodiments, a vulcanization agent used for vulcanization of rubber compounds described herein is sulfur. The sulfur is added at an amount of 1.8 parts by weight to the mixture. Heat is applied to the mixture by any known technique to activate the vulcanization process.

Any suitable vulcanization activators may be used. The vulcanization activators used may be zinc oxide and/or stearic acid which may be used independently or in any combination thereof. The activators may be added in an amount of about 1 to about 5 parts by weight and, for example, about 3 to about 5 parts by weight of zinc oxide and about 1 to about 3 parts by weight of stearic acid may be added, or any individual value or sub-range within these ranges.

Any suitable vulcanization accelerators may be used. The vulcanization accelerators used may be selected from a group consisting of guanidine, sulfonamide, thiazole and thiuram and the like, which may be used independently or in any combination thereof. These accelerators may be added in an amount of about 1.5 to about 2.5 parts by weight, or any individual value or sub-range within this range.

The vulcanization activators are, for example, added to the compound before the vulcanization process. The vulcanization agent and vulcanization accelerators are added later to avoid any premature vulcanization of the rubber compound which may cause hardening and result in reduced processability of the compound.

The rubber composition may also be selectively mixed with further components such as anti-ageing agents and anti-degradant agents.

Any suitable anti-ageing agent may be used. The anti-ageing agent used may comprise N-(1,3-dimethylbutyl)-N′-phenyl-p-phenylenediamine added in about 1 to about 3 parts by weight to the compound, or any individual value or sub-range within this range.

Any suitable anti-degradant agent may be used. The anti-degradant agent used may comprise 2,2,4-trimethyl-1,2-dihydroquinoline added in about 1 to about 3 parts by weight, or any individual value or sub-range within this range, to compounds according to embodiments herein.

Plasticizers also are used to improve the physical properties of elastomers by increasing elongation, decreasing hardness, increasing tack and improving low temperature elasticity. Plasticizers are often employed in the rubber industry because they can reduce rubber viscosity and thus improve the processing properties and filler dispersion, enhance the elasticity, and reduce processing energy consumption.

Historically, the majority of primary plasticizers have been petroleum-derived phthalates and benzoate compounds, dioctyl phthalate and diisononyl phthalate being notable examples. However, such petroleum-derived plasticizers are frequently expensive to produce and use because of fluctuations in the pricing and availability of petroleum and are increasingly likely to remain so as petroleum reserves are reduced, and new supplies prove more costly and difficult to secure. Phthalic acid esters, also named phthalates, found applications as plasticizers for the first time in 1920 and are still the largest class of plasticizers in the 21st century and particularly for polyvinylchloride (“PVC”). Nevertheless, concerns and controversy have been raised regarding the use of common plasticizers, and more specifically phthalates, for their potential to disrupt human endocrine activity, and regulatory controls have been established in a number of countries to address these concerns. Moreover, they are suspected to be bio accumulative in the environment justifying restrictive regulations in several countries regarding their use as plasticizer for flexible PVC (polyvinyl chloride) products. Both European and American regulations define six banned phthalates derivatives (diethyl hexyl phthalate (“DEHP”), dibutyl phthalate, benzyl butyl phthalate, diisobutyl phthalate (“DIBP”), di-isodecyl phthalate, and di-n-octyl phthalate) for content above 0.1 wt %.

The commercial plasticizers used in tire industry are generally petroleum-based plasticizers, such as paraffin oil, naphthenic oil and aromatic oil, which are not sustainable. Moreover, the most used aromatic oil contains a large amount of carcinogenic polycyclic aromatic hydrocarbons. Plasticizers that contain high polycyclic aromatic hydrocarbon content have been deemed carcinogenic and are effectively prohibited for use in tire production in Europe. The release of these aromatic hydrocarbon oils during the production, use and recycling of tires can cause great harm to human health and serious pollution to the environment.In addition these petroleum-based plasticizers with small molecular weight can volatilize during heat processing procedures, resulting in weak rubber mechanical properties. To reduce reliance on unsustainable crude oil, it is imperative to find environmentally friendly, renewable and harmless plasticizers for tread rubber alternatives. With the growing interest for plasticizers with low migration levels and low toxicity as an alternative to phthalates, researchers are paying more attention to bio-based plasticizers (a priori less toxic) made from vegetable oils, citrates, and sugar derivatives.

Unmodified vegetable oils are largely incompatible with PVC resin, but certain modified derivatives of vegetable oils, such as epoxidized soybean oil (“ESO”), are compatible with PVC resin and have been actively investigated for use as a lower cost, renewable source-based alternative to the petroleum-based plasticizers, both as primary and secondary plasticizers. The interest in developing useful plasticizers from renewable sources, such as vegetable oils, has developed partly also from the expectation that such materials would be less likely to cause physiological disturbances or other injuries to people coming into contact with products which require plasticizers in their composition.

### Epoxidized Alkyl Soyates and Carboxylates

Any epoxidized alkyl soyate is a particularly suitable candidate for use in one or more embodiments. It is understood that “soyate” is a carboxylate moiety which refers to any naturally occurring or subsequently refined mixture of fatty acids and their esters, where the fatty acids include stearic acid, oleic acid, linoleic acid, linolenic acid, and the like. Epoxidation of such fatty acid esters, such as methyl soyate, typically generates an epoxy group, also called a glycidyl group or oxirane ring, replacing a double bond in the fatty acid backbone.

Epoxy soyate of the formula:

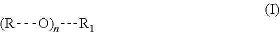

wherein n is an integer from 1 to 4,
wherein R and R₁ are independently chosen from epoxidized linoleoyl, epoxidized ricinoleoyl, epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-expoxidized myristoyl, or non-epoxidized margaroyl.

Epoxy carboxylates derived from biobased carboxylic acids of the formula

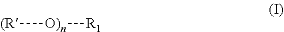

wherein R′ and R₁ are independently chosen from epoxidized dicarboxylic acyl derived from the following bio derived dicarboxylic acids:

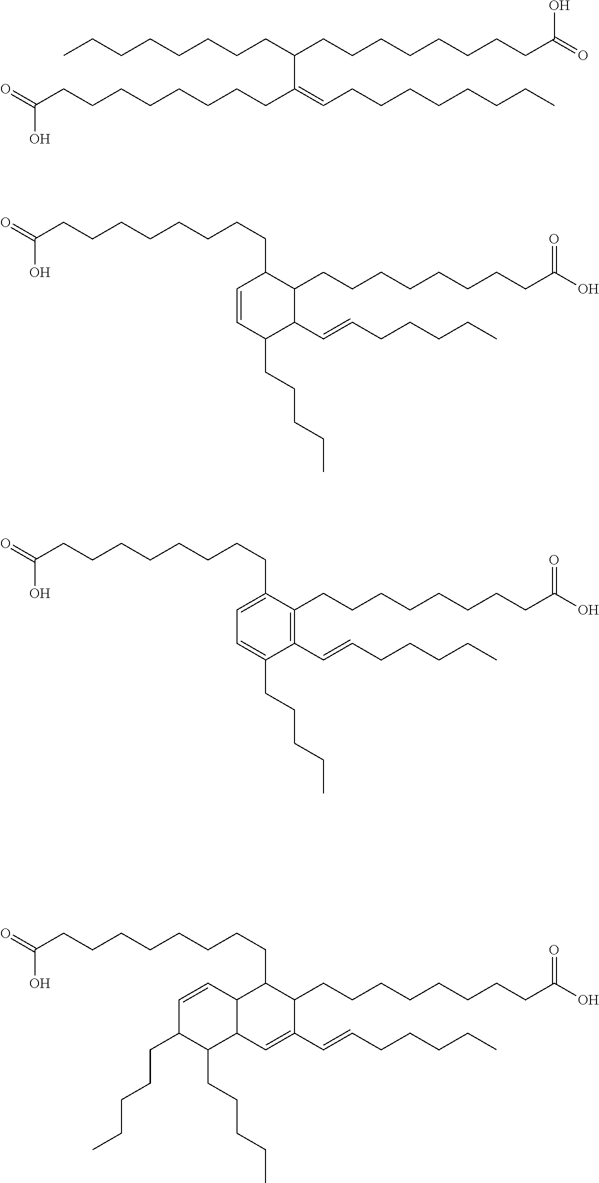

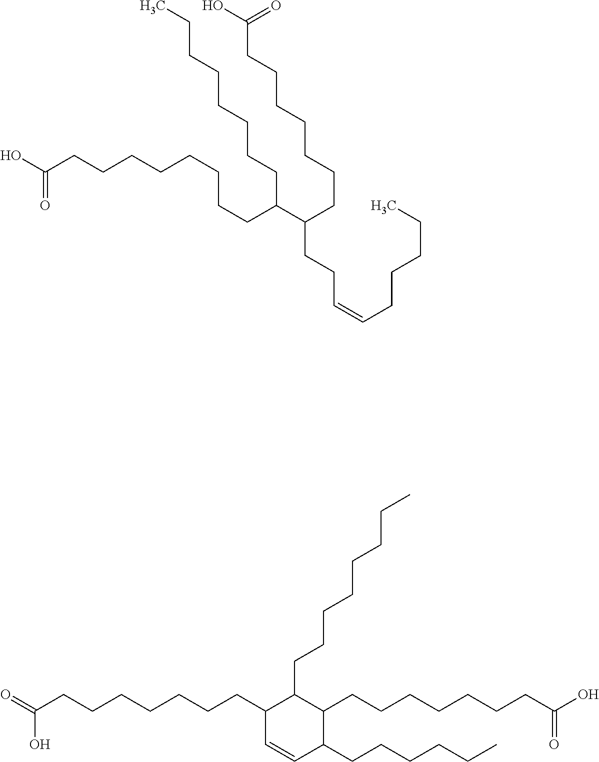

wherein, when n is 1, R₁ is alkyl or substituted of 1 to 36 carbon atoms, cycloalkyl of 5 to 12 carbon atoms, aralkyl of 7 to 9 carbon atoms or said aralkyl substituted by alkyl of 1 to 36 carbon atoms, alkoxy of 1 to 36 carbon atoms or by X, where X is a halogen atom (Cl, Br), or
wherein, when n is 2, R₁ is alkylene of 2 to 12 carbon atoms, cycloalkylene of 6 to 10 carbon atoms, arylene of 6 to 10 carbon atoms, alkylenearylenealkylene of 8 to 10 carbon atoms,
R₁ may be straight or branched chain alkyl of 1 to 36 carbon atoms such as methyl, ethyl, isopropyl, isobutyl, tert-butyl, n-octyl, 2-ethylhexyl, nonyl, n-dodecyl, n-octadecyl, eicosyl, tetracosyl, tricontyl or hexatricontyl, for example, R₁ is alkyl of 1 to 18 carbon atoms,
R₁ may alternatively be cycloalkyl of 5 to 12 carbon atoms such as cyclopentyl, cyclohexyl, cyclooctyl or cyclododecyl. For example, R₁ is cyclohexyl or cyclododecyl,
R₁ may alternatively be aralkyl of 7 to 9 carbon atoms such as benzyl, alpha-methylbenzyl, or alpha,alpha-dimethylbenzyl where the benzyl group may additionally be substituted by alkyl, for example, methyl, and where the benzyl group may additionally be substituted by alkoxy, for example, methoxy, and where the benzyl group may additionally be substituted by halogen atoms such as Cl, and/or Br.

In formula (VI), n is 1 or 2,
wherein, when n is 2, R₁ is an alkylene of 2 to 12 carbon atoms such as ethylene, 1,2-propylene, trimethylene, tetramethylene, hexamethylene, octamethylene or dodecamethylene, optionally wherein R₂ is an alkylene of 2 to 8 carbon atoms,
R₁ alternatively may be a cycloalkylene of 6 to 10 carbon atoms such as cyclohexylene, for example, 1,4-cyclohexylene, or decahydronaphthylene,
R₁ alternatively may be arylene of 6 to 10 carbon atoms such as o-, m- or p-phenylene, for example, m- or p-phenylene, or 1,4-naphthylene,
R₁ alternatively may be alkylenearylenealkylene of 8 to 10 carbon atoms, such as p-xylylene or ethylene-p-phenylene-ethylene.

Epoxy soyates and epoxy biobased carboxylates derived from sugar-based building blocks of any one of the formulas (VII)-(IX):

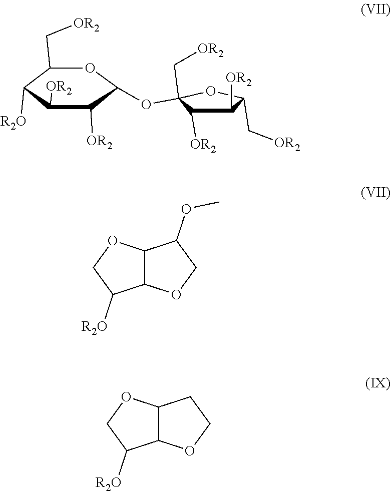

Epoxy soyates and epoxy biobased carboxylates derived from furan-based building blocks of the formula,

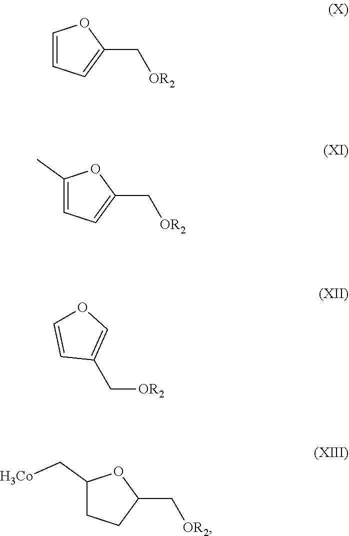

Epoxy soyates and epoxy biobased carboxylates derived from pyran-based building blocks of the formula,

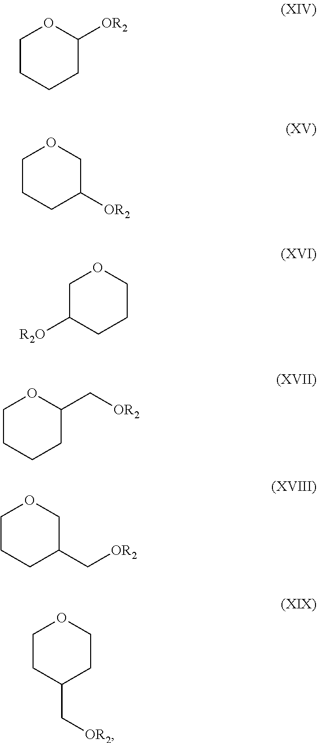

Epoxy soyates and epoxy biobased carboxylates derived from biobased dimer diol precursors.

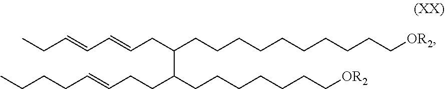

wherein each R₂ in Formulas (VII)-(XX) is independently chosen from epoxidized linoleoyl, epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-expoxidized myristoyl, and non-epoxidized margaroyl and epoxidized dicarboxylic acyl derived from the bio derived dicarboxylic acids above.

In at least one embodiment, described is a process for modifying soybean oil for use as a primary plasticizer in tire formulations. This process includes reacting fatty acids derived from vegetable oil with an alcohol (monool or polyol) to create ester linkages between the fatty acids and the alcohol by means of esterification, transesterification, or interesterification reactions, followed by epoxidation of the product of these esterification, transesterification, or interesterification reactions. Presumably, epoxidation increases the polarity and solubility parameter of the esterification, transesterification, or interesterification reaction products, resulting in increased compatibility of the vegetable-oil based plasticizer with resins used in the tire formulations. Definitional descriptions of esterification reactions, transesterification reactions, interesterification reactions, and epoxidation reactions are provided below.

Esterification is defined as the reaction of a fatty acid (e.g., carboxylic acid) with an alcohol to produce an ester and water. These reactions are equilibrium reactions and are generally driven to completion by removal of water, typically by distillation if water is the lowest boiling component. This approach was used to create the ester linkages in the following exemplary soybean oil-derived plasticizers: (i) epoxidized mono soyates (ii) epoxidized pentaerythritol tetrasoyate; (iii) epoxidized propylene glycol disoyate; and (iv) epoxidized ethylene glycol disoyate, discussed in greater detail below. The reaction below illustrates a typical esterification reaction according to embodiments herein, wherein RCO₂H is a mixture of fatty acids derived from soybean oil by hydrolysis of soybean oil, and R′OH represents alcohol functions in pentaerythritol, propylene glycol, or ethylene glycol or other suitable alcohols listed below.

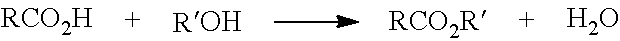

Exemplary alcohols including five to seven membered ring structures include, but are not limited to, the following: benzyl alcohol (CAS 100-51-6); 2-chlorobenzenemethanol (CAS 17849-38-6); 3-chlorobenzenemethanol (CAS 873-63-2); 4-chlorobenzenemethanol (CAS 873-76-7); 2-bromobenzenemethanol (CAS 18982-54-2); 3-bromobenzenemethanol (CAS 15852-73-0); 4-bromobenzenemethanol (CAS 873-75-6); 2-methoxybenzenemethanol (CAS 612-16-8); 3-methoxybenzenemethanol (CAS 6971-51-3); 4-methoxybenzenemethanol (CAS 105-13-5); 2-furanmethanol (CAS 98-00-0); 3-furanmethanol (CAS 143632-21-7); 5-methyl-2-furanmethanol (CAS 3857-25-8); tetrahydro-2-furanmethanol (CAS 97-99-4); tetrahydro-3-furanmethanol (CAS 15833-61-1); tetrahydro-5-(methoxymethyl)furfuryl alcohol (CAS 872303-99-6); tetrahydro-2H-pyran-2-ol (CAS 694-54-2); tetrahydro-2H-pyran-3-ol (CAS 19752-84-2); tetrahydro-2H-pyran-4-ol (CAS 2081-44-9); tetrahydro-2H-pyran-2-methanol (CAS 100-72-1); tetrahydro-2H-pyran-3-methanol (CAS 14774-36-8); tetrahydro-2H-pyran-4-methanol (CAS 14774-37-9); 1,4:3,6-dianhydro-2-O-methylhexitol (CAS 1175065-15-2) and 1,4:3,6-dianhydro-2-deoxyhexitol (CAS 1078712-23-8).

Transesterification is defined as the reaction of an ester with an alcohol to produce a derived ester and the alcohol in the original ester. These reactions are equilibrium reactions and are generally driven to completion by removal of the product alcohol, typically by distillation if this alcohol is the lowest boiling component. This approach was used to create ester linkages in several soybean oil-derived plasticizers:

The reaction below illustrates a typical transesterification reaction according to embodiments herein, wherein RCO₂R′ represents triglycerides derived from soybean or other vegetable oils, and R″OH represents pentaerythritol, propylene glycol, ethylene glycol, methanol, or other suitable alcohols listed below:

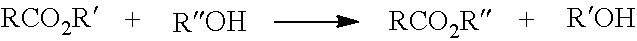

Exemplary alcohols including five to seven membered ring structures include, but are not limited to, the following: benzyl alcohol (CAS 100-51-6); 2-chlorobenzenemethanol (CAS 17849-38-6); 3-chlorobenzenemethanol (CAS 873-63-2); 4-chlorobenzenemethanol (CAS 873-76-7); 2-bromobenzenemethanol (CAS 18982-54-2); 3-bromobenzenemethanol (CAS 15852-73-0); 4-bromobenzenemethanol (CAS 873-75-6); 2-methoxybenzenemethanol (CAS 612-16-8); 3-methoxybenzenemethanol (CAS 6971-51-3); 4-methoxybenzenemethanol (CAS 105-13-5); 2-furanmethanol (CAS 98-00-0); 3-furanmethanol (CAS 143632-21-7); 5-methyl-2-furanmethanol (CAS 3857-25-8); tetrahydro-2-furanmethanol (CAS 97-99-4); tetrahydro-3-furanmethanol (CAS 15833-61-1); tetrahydro-5-(methoxymethyl)furfuryl alcohol (CAS 872303-99-6); tetrahydro-sH-pyran-2-ol (CAS 694-54-2); tetrahydro-2H-pyran-3-ol (CAS 19752-84-2); tetrahydro-2H-pyran-4-ol (CAS 2081-44-9); tetrahydro-2H-pyran-2-methanol (CAS 100-72-1); tetrahydro-2H-pyran-3-methanol (CAS 14774-36-8); tetrahydro-2H-pyran-4-methanol (CAS 14774-37-9); 1,4:3,6-dianhydro-2-O-methylhexitol (CAS 1175065-15-2) and 1,4:3,6-dianhydro-2-deoxyhexitol (CAS 1078712-23-8).

Interesterification involves the reaction of two reactant esters to produce two product esters by interchange of original alcohol functions. Again, this reaction may be driven to completion by removal of one of the product esters, typically by distillation if one of the product ester is the lowest boiling component. Interesterification is used to prepare the ester linkages in the plasticizer sucrose octasoyate by the reaction of sucrose octaacetate and methyl soyate which also produces methyl acetate that is removed by distillation. Soybean oil was also interesterified with linseed oil (with a higher IV value) to produce epoxidized, interesterified soybean oil. This interesterification process serves to increase the average number of double bonds in the modified triglyceride compared to those present in soybean oil. This significantly reduces the percentage of triglyceride molecules that have only zero, one, or two double bonds for subsequent epoxidation, thus leading to reduced migration, exudation, volatilization, and the like.

The reaction below illustrates a typical interesterification reaction according to embodiments herein, wherein RCO₂R′ represents sucrose octaacetate and R″CO₂R′″ represents methyl soyate, or alternatively wherein RCO₂R′ represents soybean oil and R″CO₂R′″ represents linseed oil.

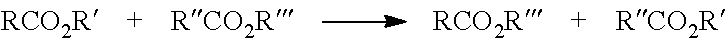

Interesterification of soybean oil with other vegetable oils results in complete randomization of all fatty acid groups present in a mixture of suitable vegetable oils. Thus, interesterification of soybean oil with a vegetable oil such as linseed oil or safflower oil, which have a higher percentage of highly unsaturated fatty acids (e.g., linolenic acid) than soybean oil, followed by epoxidation, decreases the percentage of non-epoxidized or minimally epoxidized ESO molecules. Presumably, it is these non-epoxidized or minimally epoxidized ESO molecules which are primarily responsible for exudation from tire formulations due their low solubility in or incompatibility.

In an alternative embodiment, interesterified oil is further reacted with alcohols (monools and polyols) by transesterification of the interesterified product, followed by epoxidation of the trans esterified product.

Epoxidation is defined as the addition of an oxygen atom across a carbon-carbon double bond to create epoxide (or oxirane) functionality. Epoxidation reactions are typically performed with percarboxylic acids or other peroxy compounds. The figure below illustrates a typical epoxidation reaction within

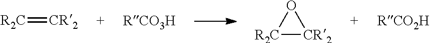

according to embodiments herein, wherein R and R′ are alkyl, substituted alkyl or hydrogen, and R″ is aryl, substituted aryl, alkyl, or hydrogen.

Suitable vegetable oil derivatives are compatible when their long chain fatty acid groups are epoxidized. According to embodiments, increasing compatibility of soybean oil by means of randomization of fatty acids, substantially complete esterification, and substantially full epoxidation results in low migration and exudation rates of this material in tire resins or matrices.

An additional reason for epoxidizing suitable vegetable-oil based plasticizers is that epoxide functionality significantly contributes to the thermal stability of the matrix. In various embodiments, vegetable oil-derivatives, which are typically useful alone as a primary plasticizer, fulfill a dual role as both the primary plasticizer and thermal stabilizer in compositions described herein.

Suitable epoxy soyates and methods of preparation thereof are described in U.S. Pat. Nos. 8,703,841 and 9,708,571 (Hagberg, et.al.,) the disclosures of which are hereby incorporated by reference in their entirety.

### Methods of Use

### Methods of Producing Tire Treads

According to various embodiments, compounds and/or compositions according to embodiments herein may be used in methods for producing rubber compounds as described. An exemplary method for producing rubber compounds includes: i) providing an appropriate amount of elastomer into a mixing device; ii) mixing the rubber polymers with a plasticizer as described herein, said neutralizing agent; and adding at a suitable time said dual-filler system in at least two separate stages; iii) adding said vulcanization agent to the mixture of step (ii); iv) heating the resultant mixture of step (iii) to vulcanize the compound.

Tires are usually made of synthetic rubber and/or natural rubber, fabric and wire, along with other compounds and chemical additives. The tire consists of a tread and a body, with the tread portion providing traction to the surface it is in contact with while the body provides support. The majority of tires today are inflatable structures where the tire is filled with compressed air to form an inflatable cushion.

It is particularly vital for tires to have good traction performance, good rolling and abrasion resistance and high wear resistance. It is impossible to have all the preceding ideal physical properties in a rubber compound. However, with the right combination of rubber components and suitable amounts of additives, a good compromise between each of the desired physical properties can be achieved.

### Manufactured Tires

Epoxy soyate plasticizers according to one or more embodiments herein are advantageous for the manufacture of motorcycle tires due to its low rolling resistance, good abrasion and wear resistance, and improved wet traction properties. This is especially useful when the tire is used on off-road surfaces, poorly maintained roads or laterite roads which are not optimal surfaces for a normal tire to operate.

A tire composition according to various embodiments has a wet traction index of 177 to 263 which aids in reducing the occurrence of aquaplaning. This usually occurs on wet roads with improper drainage design where excess amounts of water forms a layer between the tire and the road which affects the traction performance of the tires. Rubber compositions as described in one or more embodiments herein exhibit high tensile strengths of 23.1 to 27.4 MPa and elongation of 510 to 577%. Suitable rubber compounds resist wear and tear for at least a sufficient period of time before needing to change the tire. Rubber compounds according to various embodiments herein have a crescent tear value of about 107 N/mm to about 120 N/mm, or any individual value or sub-range within this range. The rubber compounds in one or more embodiments, provide high abrasion resistance and low rolling resistance. Low rolling resistance results in better fuel economy while good abrasion and wear resistance properties as well as the improved wet traction performance will aid in better tire performance compared to conventional rubber tires.

In other embodiments, a rubber composition of the present invention under ASTM D5289 exhibits Min Torque (dNm), S′ of less than 2.2, from about 1.5 to about 2.0 or about 1.8 to about 2.0; Max Torque S′ (dNm) of about 8 to about 16, about 8 to about 10 or about 13 to about 16; a T90 (min) of greater than 7 or about 7 to about 10; and/or a TS2 (min) of about 0.5 to about 5 or about 2 to about 3.

In other embodiments, a rubber composition of the present invention under ASTM D5289 exhibits Min Torque, S′ (dNm) of less than 2.2, from about 1.5 to about 2.0 or about 1.8 to about 2.0; Max Torque S′ (dNm) of about 8 to about 16, about 8 to about 10 or about 13 to about 16; a T90 (min) of greater than 7 or about 7 to about 10; and/or a TS2 (min) of about 0.5 to about 5 or about 2 to about 3.

In other embodiments, a rubber composition of the present invention under ASTM D1646 exhibits a ML (MU) of less than 55, about 30 to about 55 or about 40 to about 55; a Ts % (min) of about 15 to about 20 or about 17 to about 19; a Ts35 (min) of about 20 to about 35 or about 25 to about 30; and/or a ML, 1+4 ar 212F (MJ) of less than 65 or about 40 to about 65 or about 50 to about 65.

In other embodiments, a rubber composition of the present invention under ASTM 412A exhibits a Shore A Hardness (pts) of about 40 to about 70 ot about 50 to about 65 or about 55 to about 65; a Tensile Stregth (MPa) of about 10 to about 25 or about 14 to about 18; a M100 (MPa) of about 0.5 to about 2.5 or about 1 to about 2; a M200 (MPa) of about 2 to about 5 or about 3 to about 4; a M300 (MPa) of about 4 to about 8 or about 5 to about 7; an Ultimate Elongation (%) of about 300 to about 800, about 500 to about 700 or about 600 to about 650 and/or a Toughness (MJ/m³) of about 30 to about 60 or about 35 to about 50.

In other embodiments, a rubber composition of the present invention exhibits a Glass transition Temp (tan δ max)) (° C.) of about −25 to about −75, about −40 to about −60 or about −45 to about −55; a Glass transition Temp (E′ Onset)) (° C.) of about −50 to about −80 or about −60 to about −70; Tan 8 at 60 C of about 0.05 to about 0.3 or about 0.1 to about 0.2; Tan δ at 30 C of about 0.05 to about 0.3 or about 0.1 to about 0.2; Tan δ at 0 C of about 0.15 to about 0.4 or about 0.2 to about 0.3; Tan δ at −10 C of about 0.15 to about 0.4 or about 0.2 to about 0.3; Elastic Modulus (10⁶) (Pₐ) at 60 C of about 2 to about 10 or about 3 to about 7; Elastic Modulus (10⁶) (Pₐ) at 30 C of about 3 to about 12 or about 4 to about 9; and/or Elastic Modulus (10⁶) (Pₐ) at 0 C of about 6 to about 15 or about 7 to about 13.

In other embodiments, a rubber composition of the present invention exhibits an Abrasion Loss (mm³) under ASTM 5963 of about 40 to about 120 or about 50 to about 100 and/or an ARI (%) of about 100 to about 300 or about 125 to about 275.

### EXAMPLE(S)

The following Examples illustrate the various aspects, methods and steps of this invention. These Examples do not limit the invention, the scope of which is set out in the appended claims.

### Example 1—Preparation of 1,4-Benzenedimethanol Epoxy Soyate

A solution of soy fatty acid (1 kg, 3.66 mol), 1,4-benzenedimethanol (253 g, 1.83 mol) and p-toluene sulfonic acid monohydrate (2.1 g, 0.011 mol) in toluene was heated under reflux and water formed was continuously removed using a Dean-Stark apparatus. The reaction was monitored by titration against 0.1 N KOH in ethanol and completed at 99% conversion. (Initial AV 159/Final AV 1.2) The toluene was removed under reduced pressure and the crude product was used in the next step as is without further purification. Hydrogen peroxide (500 g, 30%) and formic acid (20 mL, 90%) were added to the crude ester and the reaction mixture was stirred at 60° C., until 1H NMR showed absence of olefinic protons indicating completion of epoxidation reaction. The reaction mixture was dissolved in dichloromethane and washed with water, brine and dried over sodium sulfate. Removal of the solvent under vacuum led to 1000 g of the title product.

### Example 2-Preparation of 4-Chlorobenzyl Epoxy Soyate

A solution of soy fatty acid (724 g, 2.65 mol), 4-chlorobenzyl alcohol (377.9 g, 2.65 mol) and p-toluene sulfonic acid monohydrate (2.1 g, 0.011 mol) in toluene was heated under reflux and water formed was continuously removed using a Dean-Stark apparatus. The reaction was monitored by titration against 0.1 N KOH in ethanol and completed at 99% conversion. (Initial AV 132/Final AV 1.1) The toluene was removed under reduced pressure and the crude product was used in the next step as is without further purification. Hydrogen peroxide (450 g, 30%) and formic acid (30 mL, 90%) were added to the crude ester and the reaction mixture was stirred at 60° C., until 1H NMR showed absence of olefinic protons indicating completion of epoxidation reaction. The reaction mixture was dissolved in dichloromethane and washed with water, brine and dried over sodium sulfate. Removal of the solvent under vacuum led to 1000 g of the title product.

### Example 3-Preparation of 4-Methylbenzyl Epoxy Soyate

A solution of soy fatty acid (724 g, 2.65 mol), 4-methylbenzyl alcohol (339.7 g, 2.78 mol) and p-toluene sulfonic acid monohydrate (1.71 g, 0.009 mol) in toluene was heated under reflux, and water formed was continuously removed using a Dean-Stark apparatus. The reaction was monitored by titration against 0.1 N KOH in ethanol and completed at 99% conversion. (Initial AV 136/Final AV 1.1) The toluene was removed under reduced pressure and the crude product was used in the next step as is without further purification. Hydrogen peroxide (450 g, 30%) and formic acid (30 mL, 90%) were added to the crude ester and the reaction mixture was stirred at 60° C., until 1H NMR showed absence of olefinic protons indicating completion of epoxidation reaction. The reaction mixture was dissolved in dichloromethane and washed with water, brine and dried over sodium sulfate. Removal of the solvent under vacuum led to 1000 g of the title product.

### Example 4-Preparation of Epoxidized Benzyl Ester of Pripol Dimer Acid

A solution of PRIPOL 1013 (1.6 kg), benzyl alcohol (600 g, 5.56 mol) and p-toluene sulfonic acid monohydrate (3 g, 0.016 mol) in toluene was heated under reflux, and water formed was continuously removed using a Dean-Stark apparatus. The reaction was monitored by titration against 0.1 N KOH in ethanol and completed at 99% conversion. (Initial AV 137/Final AV 1.0) The toluene was removed under reduced pressure and the crude product was used in the next step as is without further purification. Hydrogen peroxide (500 g, 30%) and formic acid (20 mL, 90%) were added to the crude ester and the reaction mixture was stirred at 60° C., until 1H NMR showed absence of olefinic protons indicating completion of epoxidation reaction. The reaction mixture was dissolved in dichloromethane and washed with water, brine and dried over sodium sulfate. Removal of the solvent under vacuum led to 1000 g of the title product.

### Example 5-Preparation of Blend of Epoxidized Benzyl Ester of Pripol Dimer Acid and Epoxidized 2-Ethylhexyl Ester of Dimer Acid

A solution of PRIPOL 1013 (700 g), 2-ethyl-1-hexanol (318 g, 2.44 mol) and p-toluene sulfonic acid monohydrate (1.6 g, 0.008 mol) in toluene was heated under reflux, and water formed was continuously removed using a Dean-Stark apparatus. The reaction was monitored by titration against 0.1 N KOH in ethanol and completed at 99% conversion. (Initial AV 135/Final AV 1.3) The toluene was removed under reduced pressure and the crude product was used in the next step as is without further purification. Hydrogen peroxide (200 g, 30%) and formic acid (8 mL, 90%) were added to the crude ester and the reaction mixture was stirred at 60° C., until 1H NMR showed absence of olefinic protons indicating completion of epoxidation reaction. The reaction mixture was dissolved in dichloromethane and washed with water, brine and dried over sodium sulfate. Removal of the solvent under vacuum led to 500 g of the title product.

A blend of the above with 500 g of the corresponding benzyl ester afforded 1000 g of the title blend.

### Example 6-Preparation of Epoxidized Benzyl Ester of Linseed Oil

A solution of linseed oil (1.04 kg, 3.68 mol), benzyl alcohol (400 g, 3.70 mol) and p-toluene sulfonic acid monohydrate (2.1 g, 0.011 mol) in toluene was heated under reflux, and water formed was continuously removed using a Dean-Stark apparatus. The reaction was monitored by titration against 0.1 N KOH in ethanol and completed at 99% conversion. (Initial AV 144/Final AV 1.2) The toluene was removed under reduced pressure and the crude product was used in the next step as is without further purification. Hydrogen peroxide (200 g, 30%) and formic acid (8 mL, 90%) were added to the crude ester and the reaction mixture was stirred at 60° C., until 1H NMR showed absence of olefinic protons indicating completion of epoxidation reaction. The reaction mixture was dissolved in dichloromethane and washed with water, brine and dried over sodium sulfate. Removal of the solvent under vacuum led to 1000 g of the title product.

### Example 7-Preparation of Natural Rubber Based Elastomeric Compositions Compounded with Epoxidized Soyate Esters

Elastomeric compositions were prepared for a winter tread tire formulation. The tire tread formulation contained a natural rubber based elastomeric composition according to embodiments herein mixed with epoxidized soyate. The various elastomeric compositions are shown in Table 1.

TABLE 1
Elastomeric compositions using a natural rubber based
elastomeric composition according to embodiments
herein including a an epoxidized soyate ester

| Ingredients | CONTROL | BLEND 1 | BLEND 2* |
|---|---|---|---|
| CB24¹ | 40.0 | 40.0 | 40.0 |
| SMR20² | 20.0 | 20.0 | 20.0 |
| NS116³ | 40.0 | 40.0 | 40.0 |
| Zeosil 1165MP⁴ | 75.0 | 75.0 | 75.0 |
| X50S⁵ | 12.0 | 12.0 | 12.0 |
| Sundex 8125TN⁶ | 30.0 | 30.0 | 30.0 |
| Epoxy Soyate⁷ | — | 7.5 | 7.5 |
| DPG⁸ | 2.0 | 2.0 | 2.0 |
| FPT-H Zinc⁹ | 2.00 | 2.00 | 2.00 |
| TMQ¹⁰ | 1.00 | 1.00 | 1.00 |
| Akrochem PD-2¹¹ | 1.50 | 1.50 | 1.50 |
| SA29¹² | 1.00 | 1.00 | 1.00 |
| Rubbermakers | 0.80 | 0.80 | 0.80 |
| Sulfur¹³ | | | |
| TBBS (BBTS)¹⁴ | 1.50 | 1.50 | 1.50 |

TABLE 2
Elastomeric compositions using a natural rubber based elastomeric composition
according to embodiments herein including a dual-filler system and epoxidized

| | CONTROL | BLEND | BLEND | BLEND | BLEND | BLEND | BLEND | BLEND |
|---|---|---|---|---|---|---|---|---|
| Ingredients | (A) | B | C | D | E | F | G | H |
| CB24¹ | 40.0 | 40.0 | 40.0 | 40.0 | 40.0 | 40.0 | 40.0 | 40.0 |
| SMR20² | 20.0 | 20.0 | 20.0 | 20.0 | 20.0 | 20.0 | 20.0 | 20.0 |
| NS116³ | 40.0 | 40.0 | 40.0 | 40.0 | 40.0 | 40.0 | 40.0 | 40.0 |
| Zeosil 1165MP⁴ | 75.0 | 75.0 | 75.0 | 75.0 | 75.0 | 75.0 | 75.0 | 75.0 |
| X50S⁵ | 12.0 | 12.0 | 12.0 | 12.0 | 12.0 | 12.0 | 12.0 | 12.0 |
| Sundex 8125TN⁶ | 30.0 | 30.0 | 30.0 | 30.0 | 30.0 | 30.0 | 30.0 | 30.0 |
| Example 4 | — | 7.5 | | | | | | 7.5 |
| Example 2 | | | 7.5 | | | | | |
| Example 3 | | | | 7.5 | | | | |
| Example 6 | | | | | 7.5 | | | |
| Example 1 | | | | | | 7.5 | | |
| Example 5 | | | | | | | 7.5 | |
| Benzyl Epoxy | | | | | | | | 7.5 |
| Soyate | | | | | | | | |
| DPG⁸ | 2.0 | 2.0 | 2.0 | 2.0 | 2.0 | 2.0 | 2.0 | 2.0 |
| FPT-H Zinc⁹ | 2.00 | 2.00 | 2.00 | 2.00 | 2.00 | 2.00 | 2.00 | 2.00 |
| TMQ¹⁰ | 1.00 | 1.00 | 1.00 | 1.00 | 1.00 | 1.00 | 1.00 | 1.00 |
| Akrochem | 1.50 | 1.50 | 1.50 | 1.50 | 1.50 | 1.50 | 1.50 | 1.50 |
| PD-2¹¹ | | | | | | | | |
| SA29¹² | 1.00 | 1.00 | 1.00 | 1.00 | 1.00 | 1.00 | 1.00 | 1.00 |
| Rubbermakers | 0.80 | 0.80 | 0.80 | 0.80 | 0.80 | 0.80 | 0.80 | 0.80 |
| Sulfur¹³ | | | | | | | | |
| TBBS (BBTS)¹⁴ | 1.50 | 1.50 | 1.50 | 1.50 | 1.50 | 1.50 | 1.50 | 1.50 |

*Plasticizer added in the final pass
¹CB24: Polybutadiene rubber
²SMR 20: Standard Malaysian Rubber 20
³NS116: Styrene Butadiene Rubber,
⁴Zeosil 1165MP: Amorphous precipitated silica white micropearl
⁵X50S: Bis(triethoxysilylpropyl)polysulfide (50%) carbon black (50%) filler
⁶Sundex 8125TN: Aromatic rubber process oil
⁷Epoxy soyate plasticizer according to various embodiments herein
⁸DPG (accelerator): N,N′-Diphenyl guanidine
⁹FPT-H Zinc
¹⁰TMQ (anti-degradant agent): 2,2,4-Trimethyl-1,2-dihydroquinoline
¹¹Akrochem PD-2: Phenylenediamine antiozonant
¹²SA29: Stearic acid
¹³Rubbermakers Sulfur: vulcanizing agent
¹⁴TBBS (BBTS): N-tert-butyl-2-benzothiazole sulfenamide-vulcanization accelerator

### Measurements and Tests Used

The rubber compositions were tested for the following properties:
Dispersion
Cure Characteristics (ASTM D5289)
Mooney Scorch (ASTM D1646)
Moony Viscosity (ASTM D 1646)
Tensile strength-Die B (ASTM 412A)
Hardness
Dynamic mechanical Properties
DIN Abrasion (ASTM 5963)

### Results

### Carbon Black Dispersion

All samples exhibited good dispersion.

TABLE 2
Cure Characteristics (ASTM D5289)

| MDR(160 C.) | Units | CONTROL | BLEND 1 | BLEND 2 |
|---|---|---|---|---|
| Min Torque, S′ | dNm | 2.379 | 1.805 | 2.088 |
| Max Torque S′ | dNm | 12.646 | 14.338 | 9.995 |
| T90 | min | 6.65 | 8.81 | 7.27 |
| TS2 | min | 2.23 | 2.21 | 2.61 |

TABLE 3
Cure Characteristics (ASTM D5289)

| MDR | | CONTROL | BLEND | BLEND | BLEND | BLEND | BLEND | BLEND | BLEND |
|---|---|---|---|---|---|---|---|---|---|
| (160 C.) | Units | A | B | C | D | E | F | G | H |
| Min | dNm | 2.770 | 2.216 | 2.013 | 1.878 | 1.918 | 2.117 | 2.255 | 1.821 |
| Torque, S′ | | | | | | | | | |
| Max | dNm | 12.271 | 11.469 | 13.344 | 12.448 | 13.670 | 14.603 | 11.365 | 12.327 |
| Torque S′ | | | | | | | | | |
| T90 | min | 8.80 | 8.32 | 8.79 | 9.91 | 9.99 | 9.17 | 7.92 | 11.27 |
| TS2 | min | 1.75 | 1.96 | 1.50 | 1.96 | 2.06 | 2.19 | 1.94 | 2.45 |

Min Torque, S′: Rheometer minimum torque tends to be indicative of viscosity, with a lower value indicating better performance. All blends containing epoxy soyate esters according to embodiments herein had lower minimum torque than the control.

T90: Time to 90% cure is used to establish cure times for products. Minor variations were noted between all blends containing epoxy soyate esters according to embodiments herein.

TS2: Time to 2″ inch-lb rise is indicative of scorch resistance in which a longer time is better. Addition of benzyl epoxy soyate in the final pass (Blend 2) increased scorch protection over the control. Addition of all other exoxy soyate esters according to embodiments herein, with the exception of Example 2, also increased scorch protection over the control.

TABLE 4
Mooney Scorch and Mooney Viscosity (ASTM D1646)

| | Units | CONTROL | BLEND 1 | BLEND 2 |
|---|---|---|---|---|
| Mooney Scorch | | | | |
| ML | MU | 58.66 | 40.8 | 53.62 |
| Ts5 | min | 17.3 | 17 | 19.2 |
| Ts35 | min | 27.7 | 28 | |
| Mooney Viscosity | | | | |
| ML, 1 + 4 at 212 F. | MJ | 69.9 | 49.49 | 62.56 |

TABLE 5
Mooney Scorch and Mooney Viscosity (ASTM D1646)

| | | CONTROL | BLEND | BLEND | BLEND | BLEND | BLEND | BLEND | BLEND |
|---|---|---|---|---|---|---|---|---|---|
| | Units | A | B | C | D | E | F | G | H |
| Mooney Scorch | | | | | | | | | |
| Ts5 | min | 11.4 | 13.0 | 10.9 | 14.8 | 14.5 | 16.9 | 12.6 | 19.1 |
| Mooney Viscosity | | | | | | | | | |
| ML, 1 + 4 | MJ | 70.71 | 59.05 | 54.22 | 51.30 | 51.57 | 55.81 | 58.80 | 50.31 |
| at 212 F. | | | | | | | | | |

Mooney Scorch: This is an indicator of the prevulcanization tendency of a compound, with a higher mooney scorch being better. Overall, most blends containing epoxy soyate esters according to embodiments herein were similar to the control. However, the blend containing benzyl epoxy soyate (Blend H) exhibited the longest scorch time.

Mooney Viscosity: A lower viscosity indicates better processability. All blends containing epoxy soyate esters according to embodiments herein lowered viscosity compared to the control.

TABLE 6
Tensile Properties (ASTM 412A)

| Tensile | Units | CONTROL | BLEND1 | BLEND2 |
|---|---|---|---|---|
| Shore A Hardness | pts | 60 | 63 | 54 |
| Tensile Strength | MPa | 15.6 | 17.2 | 15.2 |
| M100 | MPa | 1.7 | 1.7 | 1.4 |
| M200 | MPa | 3.7 | 3.5 | 3 |
| M300 | MPa | 6.9 | 6.1 | 5.6 |
| Ultimate elongation | % | 537.8 | 641 | 600.8 |
| Toughness | MJ/m³ | 35.83 | 48.14 | 37.73 |

TABLE 7
Tensile Properties (ASTM 412A)

| | | CONTROL | BLEND | BLEND | BLEND | BLEND | BLEND | BLEND | BLEND |
|---|---|---|---|---|---|---|---|---|---|
| Tensile | Units | A | B | C | D | E | F | G | H |
| Shore A | pts | 61 | 59 | 62 | 61 | 62 | 63 | 58 | 57 |
| Hardness | | | | | | | | | |
| Tensile | MPa | 14.4 | 14.7 | 15.5 | 15.5 | 17.2 | 17.2 | 15.1 | 16.0 |
| Strength | | | | | | | | | |
| M100 | MPa | 1.7 | 1.5 | 1.5 | 1.4 | 1.6 | 1.7 | 1.5 | 1.5 |
| M300 | MPa | 6.8 | 5.7 | 5.0 | 4.9 | 5.3 | 5.7 | 5.8 | |
| Ultimate | % | 495 | 582 | 671 | 687 | 701 | 686 | 583 | 668 |
| elongation | | | | | | | | | |
| Toughness | MJ/m³ | 29.5 | 36.3 | 46.0 | 45.8 | 52.4 | 51.3 | 37.1 | 45.1 |

Type A Hardness: Designed to an optimum level for overall product performance. All blends containing epoxy soyate esters according to embodiments herein were comparable to the control.

Tensile Strength: This is a fundamental physical property that directly influences rubber toughness, with a higher result being better. Blends containing epoxy soyate esters according to embodiments herein were slightly better than the control.

Ultimate Elongation: This is a fundamental physical property in which ahigher result is better. Blends epoxy soyate esters according to embodiments herein were much better as compared to the control.

Toughness: Calculated from the area under the stress/strain curve in which higher is better. Toughness was increased by the use of the epoxy soyate esters in accordance with embodiments herein when compared to the control.

TABLE 8
Dynamic Mechanical Properties

| Dynamic Mechanical | | | | |
|---|---|---|---|---|
| Properties | Units | CONTROL | BLEND 1 | BLEND 2 |
| Glass transition | ° C. | −49.8 | −48.8 | −49.9 |
| Temp(tan δ max) | | | | |
| Glass transition Temp | ° C. | −64.4 | −66.8 | −63.7 |
| (E′ Onset) | | | | |
| Tan δ at 60 C. | | 0.1390 | 0.1530 | 0.1540 |
| Tan δ at 30 C. | | 0.1602 | 0.1809 | 0.1781 |
| Tan δ at 0 C. | | 0.2018 | 0.2274 | 0.2277 |
| Tan δ at −10 C. | | 0.2229 | 0.2535 | 0.2473 |
| Elastic Modulus(10⁶) at | Pₐ | 6.68 | 6.51 | 3.70 |
| 60 C. | | | | |
| Elastic Modulus(10⁶) at | Pₐ | 8.24 | 8.12 | 4.62 |
| 30 C. | | | | |
| Elastic Modulus(10⁶) at | Pₐ | 12.18 | 12.87 | 7.24 |
| 0 C. | | | | |

TABLE 9
Dynamic Mechanical Properties

| Dynamic | | | | | | | | | |
|---|---|---|---|---|---|---|---|---|---|
| Mechanical | | CONTROL | BLEND | BLEND | BLEND | BLEND | BLEND | BLEND | BLEND |
| Properties | Units | A | B | C | D | E | F | G | H |
| Glass | ° C. | −47.9 | −48.7 | −48.8 | −50.2 | −50.4 | −50.3 | −47.9 | −46.9 |
| transition | | | | | | | | | |
| Temp(tan δ | | | | | | | | | |
| max) | | | | | | | | | |
| Glass | ° C. | −65.7 | −63.5 | −64.8 | −64.1 | −62.9 | −62.3 | −62.9 | −64.0 |
| transition | | | | | | | | | |
| Temp | | | | | | | | | |
| (E′ Onset) | | | | | | | | | |
| Tan δ at 60 C. | | 0.1526 | 0.1683 | 0.1871 | 0.1781 | 0.1857 | 0.1775 | 0.1672 | 0.1480 |
| Tan δ at 30 C. | | 0.1754 | 0.1907 | 0.2099 | 0.2012 | 0.2104 | 0.2041 | 0.1864 | 0.1692 |
| Tan δ at 0 C. | | 0.2084 | 0.2231 | 0.2355 | 0.2362 | 0.2442 | 0.2431 | 0.2166 | 0.2055 |
| Tan δ at −10 C. | | 0.2389 | 0.2532 | 0.2619 | 0.2597 | 0.2586 | 0.2587 | 0.2471 | 0.2400 |
| Elastic | Pₐ | 5.01 | 5.05 | 6.07 | 6.93 | 8.67 | 8.46 | 4.93 | 4.97 |
| Modulus(10⁶) | | | | | | | | | |
| at 60 C. | | | | | | | | | |
| Elastic | Pₐ | 6.42 | 6.60 | 8.24 | 9.27 | 12.11 | 11.85 | 6.38 | 6.27 |
| Modulus(10⁶) | | | | | | | | | |
| at 30 C. | | | | | | | | | |

Tan δ at 0° C.: Indicative of Wet Traction, with a higher result being better. Blends with the epoxy soyate esters according to embodiments herein were much better than the control, indicating better wet traction.

Tan δ at −10° C.: Indicative of snow/ice performance with a higher result being better. Blends with epoxy soyate esters according to embodiments herein were much better than the control, indicating better snow/ice performance.

When optimizing formulations for winter tires decreasing Tan δ at 30° C. while increasing Tan δ at −10° C. is desired. For example, blends with the epoxy soyate esters according to embodiments herein lowered Tan δ at 30° C. the most while increasing Tan δ at −10° C.

TABLE 11
DIN Abrasion(ASTM 5963)

| DIN Abrasion | Units | CONTROL | BLEND 1 | BLEND 2 |
|---|---|---|---|---|
| Abrasion Loss | mm³ | 69.9 | 57.6 | 97.6 |
| ARI | % | 213 | 259 | 152 |

TABLE 12
DIN Abrasion(ASTM 5963)

| DIN | | CONTROL | BLEND | BLEND | BLEND | BLEND | BLEND | BLEND | BLEND |
|---|---|---|---|---|---|---|---|---|---|
| Abrasion | Units | A | B | C | D | E | F | G | H |
| Abrasion | mm³ | 73.3 | 75.1 | 75.4 | 101.9 | 89.8 | 74.9 | 87.2 | 88.5 |
| Loss | | | | | | | | | |

DIN Abrasion: This is measured by resistance of a compound to abrasive sandpaper, in which a lower result is better. Improvements were observed when using the blends with epoxy soyate esters according to embodiments herein when compared to the control, indicating better abrasion resistance.

In summary, viscosity during processing and tensile and elongation of rubber formulations containing the epoxy soyate esters according to embodiments at 7.5 phr provided a desirable combination of results when compared to control. Additionally, they also demonstrated improved wet traction, snow/ice performance and abrasion.

The foregoing description discloses example embodiments of the disclosure. Modifications of the above-disclosed assemblies, apparatus, and methods which fall within the scope of the disclosure will be readily apparent to those of ordinary skill in the art. Accordingly, while the present disclosure has been disclosed in connection with example embodiments, it should be understood that other embodiments may fall within the scope of the disclosure, as defined by the following claims.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A plasticizer, comprising:
at least one compound of Formula (I): 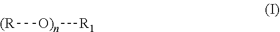
wherein:
n is an integer from 1 to 4,
R is chosen from a halogen, C₁-C₃₆ alkyl, C₁-C₃₆ alkene, C₅-C₁₂ cycloalkyl, C₇-C₉ aralkyl, C₁-C₃₆ alkoxy, epoxidized linoleoyl, epoxidized ricinoleoyl, epoxidized oleoyl, epoxidized linolenoyl, epoxidized palmitoleoyl, epoxidized dicarboxylic acyl, non-epoxidized palmitoyl, non-epoxidized stearoyl, non-epoxidized arachidoyl, non-epoxidized behenoyl, non-expoxidized myristoyl, or non-epoxidized margaroyl, wherein each of the alkyl, alkene, cycloalkyl, aralkyl, or alkoxy is straight, branched, unsubstituted, or substituted with a straight or branched C₁-C₃₆ alkyl, halogen, nitrogen or sulfur, and
R₁ is chosen from a halogen, C₁-C₃₆ alkyl, C₁-C₃₆ alkene, C₂-C₁₂ alkylene, C₆-C₁₀ cycloalkylene, C₆-C₁₀ arylene, C₈-C₁₀ alkylenearylenealkylene, C₅-C₁₂ cycloalkyl, C₇-C₉ aralkyl, C₁-C₃₆ alkoxy, epoxidized linoleoyl, epoxidized ricinoleoyl epoxidized oleoyl, epoxidized linolenoyl, epoxidized palmitoleoyl, non-epoxidized palmitoyl, non-epoxidized stearoyl, non-epoxidized arachidoyl, non-epoxidized behenoyl, non-expoxidized myristoyl, or non-epoxidized margaroyl, wherein each of the alkyl, alkene, alkylene, cyhcloalkylene, arylene, alkylenearylenealkylene, cycloalkyl, aralkyl, or alkoxy is straight, branched, unsubstituted, or substituted with a straight or branched C₁-C₃₆ alkyl, halogen, nitrogen or sulfur;
wherein Formula (I) is not benzyl epoxy soyate.

2. The compound of claim 1, wherein the epoxidized dicarboxylic acyl is derived from one of the following dicarboxylic acids:

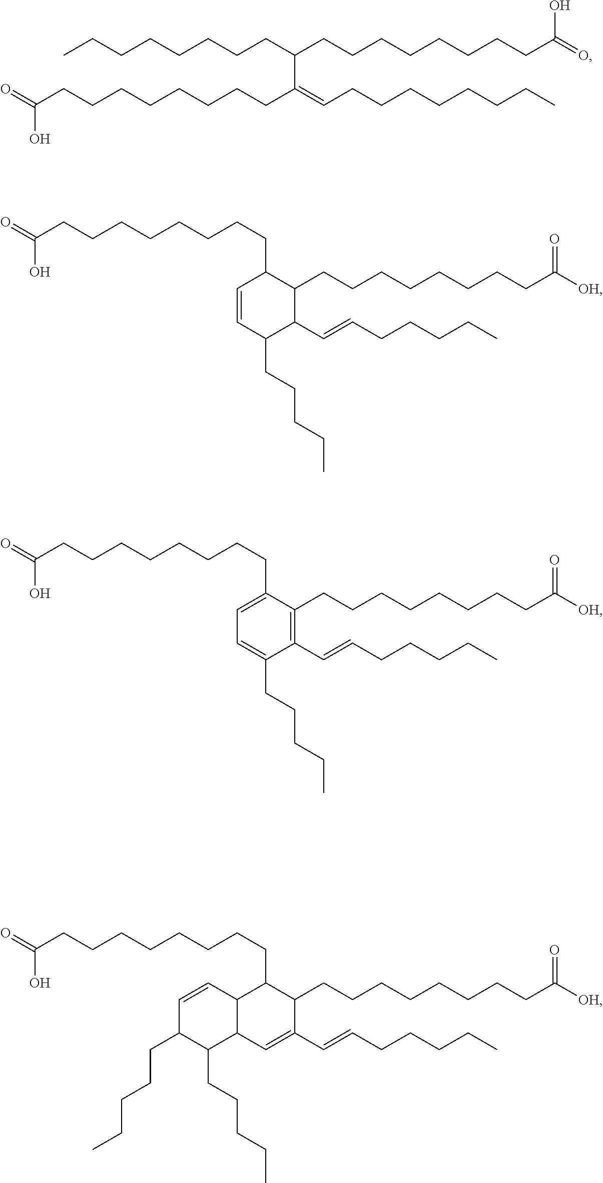 [SMILES: CCCCCCCC/C=C(/CCCCCCCCC(=O)O)C(CCCCCCCC)CCCCCCCCC(=O)O ; CCCCC/C=C/C1C(CCCCC)C=CC(CCCCCCCCC(=O)O)C1CCCCCCCCC(=O)O ; CCCCC/C=C/c1c(CCCCC)ccc(CCCCCCCCC(=O)O)c1CCCCCCCCC(=O)O ; CCCCC/C=C/C1=CC2C(C=CC(CCCCC)C2CCCCC)C(CCCCCCCCC(=O)O)C1CCCCCCCCC(=O)O]

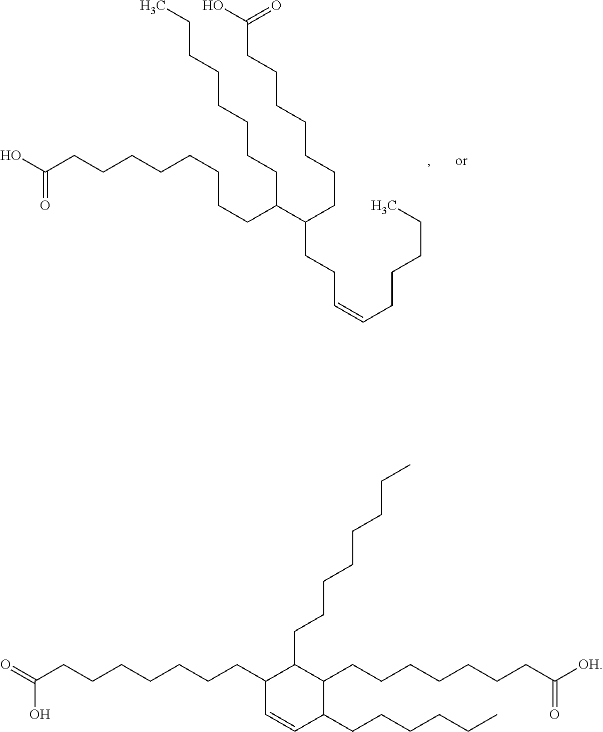 [SMILES: CCCCC/C=C\CCC(CCCCCCCC(=O)O)C(CCCCCCCC)CCCCCCCCC(=O)O ; CCCCCCCCC1C(CCCCCCCC(=O)O)C=CC(CCCCCC)C1CCCCCCCC(=O)O]

3. The plasticizer of claim 1, with the proviso that when n is 2, R₁ is the C₂-C₁₂ alkylene, C₆-C cycloalkylene, C₆-C₁₀ arylene, or C₈-C₁₀ alkylenearylenealkylene.

4. The plasticizer of claim 1, wherein R₁ is methyl, ethyl, isopropyl, isobutyl, tert-butyl, n-octyl, 2-ethylhexyl, nonyl, n-dodecyl, n-octadecyl, eicosyl, tetracosyl, tricontyl, or hexatricontyl.

5. The plasticizer of claim 1, wherein R₁ is a C₁-C₁₈ alkyl that is straight, branched, unsubstituted, or substituted with a C₁-C₃₆ alkyl, halogen, nitrogen or sulfur.

6. The plasticizer of claim 1, wherein R₁ is cyclopentyl, cyclohexyl, cyclooctyl, or cyclododecyl.

7. The plasticizer of claim 1, wherein R₁ is benzyl, alpha-methylbenzyl, or alpha, alpha-dimethylbenzyl, wherein the benzyl group is optionally substituted with a C₁-C₃₆ alkyl, a C₁-C₃₆ alkoxy, and/or a halogen.

8. The plasticizer of claim 1, wherein R1 is benzyl substituted with a methyl, a methoxy, bromine, chlorine, or any two or more thereof.

9. The plasticizer of claim 1, with the proviso that when n is 2, R₁ is a C₂-C₈ alkylene.

10. The plasticizer of claim 1, with the proviso that when n is 2, R₁ is ethylene, 1,2-propylene, trimethylene, tetramethylene, hexamethylene, octamethylene or dodecamethylene.

11. The plasticizer of claim 1, wherein R₁ is 1,4-cyclohexylene, or decahydronaphthylene.

12. The plasticizer of claim 1, wherein R₁ is an o-, m- or p-phenylene.

13. The plasticizer of claim 1, wherein R₁ is 1,4-naphthylene.

14. The plasticizer of claim 1, wherein R₁ is p-xylylene, or ethylene-p-phenylene-ethylene.

15. The plasticizer of claim 1, wherein the compound is 4-chlorobenzyl epoxy soyate.

16. (canceled)

17. The plasticizer of claim 1, wherein the compound is 1,4-benzenedimethanol epoxy soyate.

18-39. (canceled)

40. A tire, comprising: a plasticizer according to claim 1.

41. The tire of claim 40, wherein the tire exhibits at least one of: low rolling resistance, good abrasion, good wear resistance, or improved wet traction performance.

42. A rubber, comprising: a plasticizer according to claim 1.

43. A rubber component, comprising: a plasticizer according to claim 1.

44. The rubber component of claim 43, wherein the plasticizer comprises 4-chlorobenzyl epoxy soyate, 4-methyl benzyl epoxy soyate, 1,4-benzenedimethanol epoxy soyate, epoxidized benzyl ester of linseed oil, epoxidized benzyl ester of dimer C36 acid, epoxidized 2-ethylhexyl ester of dimer C36 acid, 50/50 blend of epoxidized 2-ethylhexyl ester of dimer C36 acid and epoxidized benzyl ester of dimer C36 acid.

45. The rubber component of claim 43 wherein the rubber component is a mat, conveyor belt, drive belt, drive rollers, hose, rubber articles of food processing equipment, rubber articles of sports equipment, gloves, rubber flooring, shoe, wet suit, clothing, playground equipment, industrial roller, or seal.

46-48. (canceled)
